(19) United States
(12) Patent Application Publication
(10) Pub. No.: US 2013/0029955 A1
(43) Pub. Date: Jan. 31, 2013

(54) INHIBITORS OF GALECTIN-3 AND METHODS OF USE THEREOF

(71) Applicant: Pieter Muntendam, Boxford, MA (US)

(72) Inventor: Pieter Muntendam, Boxford, MA (US)

(21) Appl. No.: 13/480,077

(22) Filed: May 24, 2012

Related U.S. Application Data
(60) Provisional application No. 61/490,049, filed on May 25, 2011.

(51) Int. Cl. A61K 31/585; A61K 31/7016; G06Q 50/22; G01N 33/566; G01N 21/64; A61K 31/70; A61K 31/732

(52) U.S. Cl. 514/173; 514/23; 514/53; 514/54; 436/501; 435/7.94; 435/7.1; 705/2

(57) ABSTRACT

Described herein are methods and compositions for inhibiting galectin-3 in a subject. Also described are methods and compositions for treating heart failure.

### REFERENCE TO RELATED APPLICATIONS

This application claims the benefit of and priority to U.S. Provisional Patent Application No. 61/490,049, filed May 25, 2011, the complete disclosure of which is incorporated by reference herein.

### BACKGROUND OF THE INVENTION

Heart failure (HF) is a major public health problem in the United States. Approximately 5 million people suffer from the disease and the number of patients is steadily increasing. HF is a common but severe and complex clinical syndrome, especially among elderly people. HF refers to a condition in which the heart fails to pump enough blood to meet the body's needs. Insufficient pumping leads to the congestion of blood and other fluid in the liver, abdomen, lower extremities, and lungs. Thus, HF has also been called congestive heart failure (CHF), although the term HF is preferred because not all patients exhibit fluid congestion. HF results in a gradual deterioration of the patient often leading to cardiovascular mortality. Thus, a large number of patients die within one to five years after diagnosis. However, others may remain stable for prolonged periods. HF is a very different disease from heart ischemia caused by myocardial infarction or reperfusion injury.

Symptoms of HF include fatigue, weakness, rapid or irregular heartbeat, shortness of breath, persistent cough or wheezing, swelling of lower extremities or abdomen, sudden weight gain from fluid retention, lack of appetite or nausea, and chest pain. Patients with or at risk of developing HF may also display increased or decreased levels of certain biomarkers. Blood tests for biomarkers, such as B-type natriuretic peptide (BNP) and galectin-3, can be performed and are good indicators of heart failure.

Galectins constitute a family of proteins characterized by their galactose-specific binding. All share common amino acid sequence in regions of their structure known as the carbohydrate recognition domain or CRD. Currently, 15 mammalian galectins have been identified. One subgroup contains galectins 1, 2, 5, 7, 10, 13, 14, and 15, each of which comprises a single CRD. A second subgroup contains a single species, galectin-3, which comprises a single CRD linked to an N-terminal domain comprising repeats of short amino acid sequences such as PGA. A third galectin subgroup contains galectins 4, 6, 8, 9, and 12, each of which comprises two CRDs joined by a linker of variable length. All of the galectins have significant amino acid sequence homology, and many appear in the human circulatory system; however, galectin-3 is currently the only galectin having a known relation with HF.

### SUMMARY OF THE INVENTION

Described herein are methods and compositions for inhibiting galectin-3 in a subject. Also described are methods and compositions for treating heart failure.

In one aspect, a method for treating heart failure in a patient is provided. The method comprises administering to the patient a pharmaceutical composition comprising a carbohydrate in an amount sufficient to at least partially alleviate a symptom of heart failure, wherein the carbohydrate binds to galectin-3.

In another aspect, a method for treating a patient at risk of developing heart failure is provided. The method comprises administering to the patient a pharmaceutical composition comprising a carbohydrate in an amount sufficient to reduce the risk of developing heart failure, wherein the carbohydrate binds to galectin-3.

In yet another aspect, a method for treating heart failure in a patient is provided. The method comprises identifying the patient on the basis that the patient has elevated circulatory levels of galectin-3 and administering to the patient a compound in an amount sufficient to prevent further decrease, or to increase, left ventricular ejection fraction, wherein the compound binds galectin-3.

In still another aspect, a method for treating heart failure in a patient is provided. The method comprises identifying the patient on the basis that the patient has elevated circulatory levels of galectin-3 and administering to the patient a compound in an amount sufficient to decrease left ventricular end diastolic pressure, wherein the compound binds galectin-3.

In yet another aspect, a method for treating heart failure in a patient is provided. The method comprises identifying the patient on the basis that the patient has elevated circulatory levels of galectin-3 and administering to the patient a compound in an amount sufficient to at least partially inhibit cardiac remodeling, wherein the compound binds galectin-3.

In still another aspect, a method for treating heart failure in a patient is provided. The method comprises identifying the patient on the basis that the patient has elevated circulatory levels of galectin-3 and administering to the patient a compound in an amount sufficient to at least partially inhibit cardiac fibrosis, wherein the compound binds galectin-3.

In yet another aspect, a method of inhibiting galectin-3 in a patient in need thereof is provided. The method comprises administering to the patient a composition comprising a carbohydrate and a pharmaceutically acceptable carrier, wherein the carbohydrate binds to galectin-3 and inhibits an activity of galectin-3 and determining the activity of galectin-3 in the patient.

In still another aspect, a method of inhibiting galectin-3 in a patient in need thereof is provided. The method comprises administering to the patient an ingestible composition comprising a compound that binds galectin-3 and inhibits an activity of galectin-3 and determining the activity of galectin-3 in the patient.

In yet another aspect, a composition is provided. The composition comprises a purified pectin fragment and a pharmaceutically acceptable carrier, wherein the purified pectin fragment is capable of binding to galectin-3 and inhibiting an activity of galectin-3.

In still another aspect, a method of inhibiting galectin-3 in a patient in need thereof is provided. The method comprises administering to the patient a composition comprising a purified pectin fragment and a pharmaceutically acceptable carrier, wherein the purified pectin fragment binds to galectin-3 and inhibits an activity of galectin-3.

In yet another aspect, a method of determining a galectin-3 inhibitory activity of a foodstuff is provided. The method comprises administering the foodstuff to a subject and determining the activity of galectin-3 in the subject after administering the foodstuff to the subject.

In still another aspect, a method for providing information relating to galectin-3 inhibiting effects of a food product to a user is provided. The method comprises providing a server having a network interface and hosting characteristics relating to a number of food products, soliciting a user to input at least one food product characteristic to the server, comparing the user input food product characteristic with the hosted food product characteristics, selecting at least one hosted food product with characteristics comparable to the at least one user input characteristic, and presenting to the user information relating to the galectin-3 inhibiting effects of the at least one selected food product.

In yet another aspect, a method of selecting a therapy for a human is selected. The method comprises measuring a galectin-3 blood concentration in a sample from the human, thereby to determine the presence or absence of a galectin-3 blood concentration indicative of responsiveness to an aldosterone antagonist.

In still another aspect, a method of treating a human is provided. The method comprises repeatedly administering an aldosterone antagonist to a patient having a determined galectin-3 blood concentration indicative of a survival-enhancing response to the aldosterone antagonist.

### DESCRIPTION OF THE DRAWINGS

FIG. 1 is a flow chart of the operation of a server, according to an embodiment;

FIG. 2 is a depiction of a server, according to an embodiment;

FIG. 3A is a bar graph showing fractional shortening for each study group of rats, according to an embodiment;

FIG. 3B is a plot showing fractional shortening for each study group of rats at the beginning of the study and at sacrifice, according to an embodiment;

FIG. 3C is a bar graph showing left ventricle end diastolic pressure (LVEDP) for each study group of rats, according to an embodiment;

FIG. 3D is a bar graph showing left ventricle pressure decay (Tau) for each study group of rats, according to an embodiment;

FIG. 4A shows micrographs of cardiac tissue sections for each study group of rats, according to an embodiment;

FIG. 4B is a bar graph that quantifies fibrosis for each study group of rats, according to an embodiment; and

FIG. 5 is a plot of study group survival as a function of time, according to an embodiment.

Other aspects, embodiments, and features will be apparent from the following detailed description when considered in conjunction with the accompanying figures.

### DESCRIPTION OF THE INVENTION

Described herein are methods and compositions for inhibiting galectin-3 in a subject. Also described are methods and compositions for treating heart failure. Various compositions comprising a compound that binds to galectin-3 are provided. In some embodiments, a composition may comprise a carbohydrate. For example, the composition may comprise a pectin and/or a purified pectin fragment. In another embodiment, the composition may comprise a substituted lactosamine (e.g., N-acetyllactosamine). In one aspect, the composition may be administered to a subject. In some embodiments, the composition may inhibit an activity of galectin-3. In certain embodiments, the activity of galectin-3 may be determined in a subject before and/or after administration of a composition comprising a compound that binds galectin-3. In another aspect, methods of treating a subject having or at risk of developing heart failure are provided. In some embodiments, a symptom of heart failure may be at least partially alleviated. Also described are methods for determining a galectin-3 inhibitory activity of a foodstuff.

The terms “heart failure,” “HF,” “congestive heart failure, or “CHF” as used herein, refer to the complex clinical syndrome that impairs the ability of the ventricle to fill with or eject blood. Any structural or functional cardiac disorder can cause HF, with the majority of HF patients having impaired left ventricular (LV) myocardial function. Outward symptoms of HF include dyspnea (shortness of breath), fatigue, and fluid retention. Internal symptoms of heart failure include cardiac fibrosis, cardiac remodeling, depressed fractional shortening, increased left ventricle end diastolic pressure (LVEDP), elevated right ventricular end diastolic pressure (RVEDP), depressed left ventricular ejection fraction, depressed left ventricular end diastolic volume, depressed left ventricular right systolic volume, and increased left ventricle relaxation time constant (Tau).

The American Heart Association (AHA) has identified 4 stages in the development of HF. Patients in stages A and B show clear risk factors but have not yet developed HF. Patients in stages C and D currently exhibit or in the past have exhibited symptoms of HF. For example, Stage A patients are those with risk factors such as coronary artery disease, hypertension or diabetes mellitus who do not show impaired left ventricular (LV) function. Stage B patients are asymptomatic, but have cardiac structural abnormalities or remodeling, such as impaired LV function, hypertrophy or geometric chamber distortion. Stage C patients have cardiac abnormalities and are symptomatic. Stage D patients have refractory HF in which they exhibit symptoms despite maximal medical treatment. They are typically recurrently hospitalized or unable to leave the hospital without specialized intervention.

Galectin-3 (GenBank Accession Nos.: NC_{—}000014.7 (gene) and NP_{—}002297.2 (protein)) is one of 15 mammalian beta galactoside-binding lectins, or “galectins,” characterized by their galactose-specific binding. Galectin-3 has variously been referred to in the literature as LGALS3, MAC-2 antigen, Carbohydrate binding protein (CBP)-35, laminin binding protein, galactose-specific lectin 3, mL-34, L-29, hL-31, epsilon BP, and IgE-binding protein. Galectin-3 is composed of a carboxyl-terminal carbohydrate recognition domain (CRD) and amino-terminal tandem repeats (Liu, F.-T. (2000) Role of galectin-3 in inflammation. In Lectins and Pathology. M. Caron and D. Seve, eds. Harwood Academic Publishers, Amsterdam, The Netherlands, p. 51; Liu, F.-T. et al. (1995) Am. J. Pathol. 147:1016). Galectin-3 normally distributes in epithelia of many organs and various inflammatory cells, including macrophages as well as dendritic cells and Kupffer cells (Flotte, T. J. et al. (1983) Am. J. Pathol. 111:112).

Galectin-3 has been shown to play a role in a variety of cellular process, including cell-cell adhesion, cell-matrix interactions, phagocytosis, cell cycle, apoptosis, angiogenesis and mRNA splicing. Galectin-3 has been shown to function through both intracellular and extracellular actions (Sano, H. et al. (2000) The Journal of Immunology, 165:2156-2164). It is a component of heterogeneous nuclear ribonuclear protein (hnRNP) (Laing, J. G. et al. (1998) Biochemistry 27:5329), a factor in pre-mRNA splicing (Dagher, S. F. et al. (1995) Proc. Natl. Acad. Sci. USA 92:1213) and has been found to control the cell cycle (Kim, H.-R. C. et al. (1999) Cancer Res. 59:4148) and prevent T-cell apoptosis through interaction with the Bcl-2 family members (Yang, R.-Y. et al. (1996) Proc. Natl. Acad. Sci. USA 93:6737). On the other hand, galectin-3, which is secreted from monocytes/macrophages (Sato, S. et al. (1994) J. Biol. Chem. 269:4424) and epithelial cells (Lindstedt, R. G. et al. (1993) J. Biol. Chem. 268:11750) has been demonstrated to function as an extracellular molecule in activating various types of cells such as monocytes/macrophages (Liu, F.-T. (1993) Immunol Today 14:486), mast cells, neutrophils and lymphocytes (Hsu, D. K., S. R. et al. (1996). Am. J. Pathol. 148:1661). Galectin-3 has been shown to act as a novel chemoattractant for monocytes and macrophages (Sano, H. et al. (2000) The Journal of Immunology, 2000, 165:2156-2164). Galectin-3 has been implicated in diseases and conditions such as cancer, inflammation, and heart failure. As disclosed in International Patent Publication No. WO2005/040817, quantitation of galectin-3 is particularly suitable for use in an assay to diagnose and detect the severity of HF and to predict outcome.

### Compositions

In some embodiments, a composition may comprise a compound that is capable of binding galectin-3. Any suitable compound may be used. For example, in certain embodiments, the compound may be a carbohydrate, a protein (e.g., an antibody or antibody fragment), a nucleic acid (e.g., an aptamer), or a small molecule.

In some embodiments, the compound may be a carbohydrate. For example, the compound may be a polysaccharide, a disaccharide, a monosaccharide, a pectin, a naturally-occurring carbohydrate, a synthetic carbohydrate, and the like. Pectins are polysaccharides found in the cell walls of terrestrial plants. In some embodiments, a pectin may be a full-length pectin, e.g., a pectin that has not been subjected to fragmentation. In other embodiments, the pectin may be a pectin fragment. In some instances, a pectin may be linear. In other instances, a pectin may be branched. In some cases, the pectin may be a homogalacturonan, a substituted galacturonan, or a rhamnogalacturonan. In some embodiments, a pectin may comprise galactose, xylose, apiose, glucose, arabinose, rhamnose, uronic acid (e.g., galacturonic acid) and/or mannose residues. In some embodiments, a pectin may be a mixture of chemical species. For example, a pectin may comprise a molecular weight distribution of polysaccharide chains. In some instances, a pectin may comprise two or more polysaccharides of different chemical composition.

In some embodiments, a homogalacturonan may comprise a linear chain of galacturonic acid (e.g., a-(1-4)-linked D-galacturonic acid). A substituted galacturonan may, in some instances, comprise a backbone of galacturonic acid residues (e.g., galacturonic acid) with at least some of the backbone residues substituted with pendant side groups of saccharide residues. In some embodiments, a side group may comprise xylose, galactose, apiose, glucose, arabinose, mannose, and/or combinations thereof.

In some cases, a pectin may be a rhamnogalacturonan pectin. For example, the rhamnogalacturonan may be a rhamnogalacturonan I pectin or a rhamnogalacturonan II pectin. A rhamnogalacturonan I pectin may have, in some embodiments, a backbone of repeating galacturonic acid-rhamnose disaccharides (e.g., α-D-galacturonic acid-(1,2)-α-L-rhamnose). In some cases, rhamnogalacturonan II may have a backbone that is essentially all galacturonic acid residues (e.g., D-galacturonic acid). In some embodiments, at least some of the backbone residues may be substituted with pendant side groups of saccharide residues. In some embodiments, a side group may comprise xylose, apiose, glucose, arabinose, or mannose.

In some cases, a side group may include only one residue species. In other cases, a side group may include a mixtures of residues. It should be understood that the saccharide residues in a pectin may comprise L isomers, D isomers, or a mixture of L isomers and D isomers. In some embodiments, the pectin may comprise alpha linkages, beta linkages, or a combination of alpha and beta linkages. In some embodiments, a side group may be one or more residues in length. For example, a side group may have a length of 1 residue, 2 residues, 3 residues, 4 residues, 5 residues, 10 residues, 15 residues, 20 residues, 30 residues, 50 residues, or even more.

As would be understood by one of ordinary skill in the art, multimers of saccharides may be referred to by chemical names such as galactan (i.e., a multimer of galactose units), arabinogalactan (i.e., a multimer of arabinose and galactose units), arabinan (i.e., a multimer of arabinose units), and rhamnogalacturonan (i.e., a multimer of rhamnose and galacturonic acid units).

In some embodiments, a functional group of a pectin may be modified. For example, in some embodiments, at least some of the carboxyl groups of galacturonic acid may be esterified. In some instances, at least some of the carboxyl groups may be alkyl methyl esters (e.g., methyl esters). In some embodiments, more than half of the carboxyl groups in a pectin may be esterified (i.e., the pectin may be a “high-ester pectin”). In other embodiments, less than half of the carboxyl groups in a pectin may be esterified (i.e., the pectin may be a “low-ester pectin”). In some cases, between about 10% and about 90% of the carboxyl groups may be esterified. In some embodiments, a pectin may be acetylated. In other embodiments, a pectin may be amidated.

In some embodiments, a pectin may have a molecular weight of between about 50 kDa and about 150 kDa, between about 60 kDa to about 130 kDa, between about 50 kDa and about 100 kDa, between about 30 kDa and about 60 kDa, between about 10 kDa and about 50 kDa, between about 10 kDa and about 30 kDa, between about 10 kDa and about 20 kDa, between about 5 kDa and about 20 kDa, or between about 1 kDa and about 10 kDa.

In some embodiments, a pectin may be obtained from a natural source. For instance, in some cases, a pectin may be obtained from a plant source. Non-limiting examples of plant sources include fruits (e.g., apples, guavas, quince, pears, plums, gooseberries, oranges, lemons, grapefruits, other citrus fruits, cherries, grapes, strawberries, and the like) and vegetables (e.g., sugar beets, potatoes, and carrots), although any suitable source may be utilized. In some embodiments, a pectin may be obtained from citrus peel. In other embodiments, a pectin may be obtained from apple pomace. In some embodiments, a pectin may be a swallow root pectic polysaccharide, Hemidesmus pectic polysaccharide, black cumin pectic polysaccharide, Andrographis pectic polysaccharide, citrus pectic polysaccharide, or modified swallow root pectic polysaccharide.

In some embodiments, a pectin may be fragmented into two or more fragments. For example, a pectin may be fragmented by exposure to any suitable chemical condition to form a pectin fragment. For example, in some cases, a pectin may be fragmented by hydrolysis (e.g., acid hydrolysis, alkaline hydrolysis, and/or catalytic hydrolysis), enzymatic digestion, oxidative lysis, and/or radiative lysis (i.e., by x-rays or gamma rays). Generally, “pectin fragment” refers to a pectin having a molecular weight less than the parent pectin from which the pectin fragment is derived. However, it should be understood that a pectin fragment may be subjected to modification that may alter the molecular weight.

Pectin fragments of interest are capable of binding to galectin-3. In some instances, a pectin fragment may bind to galectin-3 more strongly than the parent pectin from which the pectin fragment was derived. In some embodiments, it may be advantageous to purify a pectin fragment. For example, a mixture of pectin fragments may contain fragments that bind to galectin-3 and fragments that do not bind to galectin-3. Purifying the mixture such that it contains a higher proportion of fragments that bind galectin-3 may, in some embodiments, result in such advantageous properties as increased efficacy and/or reduced side effects upon administration to a subject.

A pectin fragment may be purified by any suitable method. For example, in some embodiments, a pectin fragment may be purified with respect to molecular weight. In some cases, certain molecular weight fractions may have enhanced galectin-3 binding affinity. In some embodiments, fractions with enhanced galectin-3 binding affinity may be identified by subjected the fractions to a galectin-3 binding assay, as described in more detail herein. In another example, a pectin fragment may be purified by affinity chromatography. The affinity chromatography resin may comprise, in some embodiments, galectin-3, a galectin-3 fragment, or any material that mimics a galectin-3 binding site.

As discussed above, a galectin-3 inhibitor may be a carbohydrate, such as a monosaccharide, a disaccharide, a trisaccharide, a polysaccharide, or analogs or derivatives thereof. Any suitable carbohydrate may be used. In some embodiments, the galectin-3 inhibitor may comprise galactose. In some embodiments, the galectin-3 inhibitor may comprise glucose, galactose, fucose, arabinose, arabitol, allose, altrose, gulose, galactosamine, hammelose, lyxose, mannose, mannitol, mannosamine, ribose, rhamnose, threose, talose, xylose, uronic acids thereof, and combinations thereof. Non-limiting examples of carbohydrates include lactose; LacNAc; Gal-β-1,4-GlcNAc-β-1,3-Gal-β1,4-Glc; Gal-β-1,3-GlcNAc-β-1,3-Gal-β-1,4-Glc; Gal-β-1,4-GlcNAc-β-1,3-Gal; Gal-β-1,4-GlcNAc-β-1,2-(Gal-β-1,4-GlcNAc-β-1,6)-Man; Me-β-LacNAc; Gal-β-1,4-GlcNAc-β-1,2-(Gal-β-1,4-GlcNAc-β-1,4)-Man-α-1,3)-(Gal-β-1,4-GlcNAc-β-1,2-(Gal-β-1,4-GlcNAc-β-1,6)-Man-α-1,6)-Man; Gal-β-1,4-Fru; Gal-β-1,4-ManNAc; Gal-α-1,6-Gal; Me-β-Gal; GlcNAc-β-1,3-Gal; GlcNAc-β-1,4-GlcNAc; Glc-β-1,4-Glc; and GlcNAc; where Gal is galactosyl, Glc is glucosyl, Man is mannosyl, Fm is fructosyl, NAc is N-acetyl, and Me is methyl.

In some embodiments, a compound (e.g., a carbohydrate) may be derivatized with one or more substituents. For example, a compound may be derivatized with one or more substituents, where each substituent may independently be a substituted or unsubstituted aliphatic, substituted or unsubstituted heteroaliphatic, substituted or unsubstituted cycloalkyl, substituted or unsubstituted heterocyclic, substituted or unsubstituted aryl, substituted or unsubstituted heteroaryl, or acyl group. In some embodiments, a carbohydrate may be N-substituted. In another embodiments, a carbohydrate may be O-substituted.

In certain embodiments, the carbohydrate may be a substituted lactosamine. For example, the carbohydrate may be an N-substituted lactosamine (e.g., LacNAc).

In some embodiments, a carbohydrate may be resistant to metabolism. For example, in some instances, a carbohydrate may include a thio linkage between a first saccharide unit and a second saccharide unit, which impairs hydrolysis of the saccharide-saccharide bond.

In some cases, an inhibitor of galectin-3 activity may include a glycoconjugate, such as a glycolipid, glycopeptide, or proteoglycan.

In some embodiments, an ingestible composition may comprise a compound capable of inhibiting galectin-3. For example, the ingestible composition may be a foodstuff. As discussed in more detail below, a foodstuff may be screened to determine the ability of the foodstuff to inhibit galectin-3. In some cases, the foodstuff may contain a pectin. For example, the foodstuff may be a fruit and/or vegetable product, such as a baked good, a beverage, a mixture of raw and/or cooked fruits and/or vegetables, and the like. In certain embodiments, a foodstuff may be fortified with a compound capable of binding to galectin-3. In some cases, the foodstuff may be fortified with an amount of a compound capable of binding to galectin-3 that is sufficient to have a therapeutic (e.g., cardiotherapeutic and/or cardioprotective) effect on a subject. Such an approach may be particularly advantageous for improving a foodstuff having non-therapeutically relevant amounts or essentially no amount of a galectin-3 inhibitor.

Additional inhibitors of galectin-3 include nucleic acids, such as antisense nucleic acids and nucleic acid aptamers. Antisense nucleic acids refers to a polynucleotide or peptide nucleic acid capable of binding to a specific DNA or RNA sequence and inhibiting galectin-3 expression. In some embodiments, an antisense nucleic acid may be targeted to a region of the gene encoding galectin-3 in a cell.

In some embodiments, a galectin-3 antibody may be used as an inhibitor of galectin-3. In some cases, the antibody may be selective for an epitope present in galectin-3. Further embodiments are described in more detail below.

In some instances, a human or humanized antibody may be used. The term “human” when used in reference to an antibody, means that the amino acid sequence of the antibody is fully human. A “human galectin-3 antibody” or “human anti-galectin-3 antibody” therefore refers to an antibody having human immunoglobulin amino acid sequences, i.e., human heavy and light chain variable and constant regions that specifically bind to galectin-3. That is, all of the antibody amino acids are human or exist in a human antibody. An antibody that is non-human may be made fully human by substituting the non-human amino acid residues with amino acid residues that exist in a human antibody. The term “humanized antibody”, as used herein, refers to antibody molecules in which amino acids have been replaced in the non-antigen binding regions in order to more closely resemble a human antibody, while still retaining the original binding ability. The term “humanized” therefore means that the amino acid sequence of the antibody has non-human amino acid residues (e.g., mouse, rat, goat, rabbit, etc.) of one or more determining regions (CDRs) that specifically bind to the desired antigen (e.g., galectin-3) in an acceptor human immunoglobulin molecule, and one or more human amino acid residues in the Fv framework region (FR), which are amino acid residues that flank the CDRs. Human framework region residues of the immunoglobulin can be replaced with corresponding non-human residues. Residues in the human framework regions can therefore be substituted with a corresponding residue from the non-human CDR donor antibody to alter, generally to improve, antigen affinity or specificity, for example. In addition, a humanized antibody may include residues, which are found neither in the human antibody nor in the donor CDR or framework sequences. For example, a framework substitution at a particular position that is not found in a human antibody or the donor non-human antibody may be predicted to improve binding affinity or specificity human antibody at that position. Antibody framework and CDR substitutions based upon molecular modeling are well known in the art, e.g., by modeling of the interactions of the CDR and framework residues to identify framework residues important for antigen binding and sequence comparison to identify unusual framework residues at particular positions. Antibodies referred to as “primatized” in the art are within the meaning of “humanized” as used herein, except that the acceptor human immunoglobulin molecule and framework region amino acid residues may be any primate residue, in addition to any human residue.

The term “binding specificity,” when used in reference to an antibody, means that the antibody specifically binds to all or a part of the same antigenic epitope or sequence as the reference antibody. A part of an antigenic epitope or sequence means a subsequence or a portion of the epitope or sequence. For example, if an epitope includes 8 contiguous amino acids, a subsequence and, therefore, a part of an epitope may be 7 or fewer amino acids within this 8 amino acid sequence epitope. In addition, if an epitope includes non-contiguous amino acid sequences, such as a 5 amino acid sequence and an 8 amino acid sequence which are not contiguous with each other, but form an epitope due to protein folding, a subsequence and, therefore, a part of an epitope may be either the 5 amino acid sequence or the 8 amino acid sequence alone.

Galectin-3 antibodies include human, humanized, and chimeric antibodies. For example, a galectin-3 antibody may have a dissociation constant (Kd) less than about 10⁻⁴ M, less than about 10⁻⁵ M, less than about 10⁻⁶ M, less than about 10⁻⁷ M, or less than about 10⁻⁸ M.

Methods of producing human antibodies are known in the art. For example, human transchromosomic KM Mice™ (WO 02/43478) and HAC mice (WO 02/092812) express human immunoglobulin genes. Using conventional hybridoma technology, splenocytes from immunized mice that respond to galectin-3 can be isolated and fused with myeloma cells. An overview of the technology for producing human antibodies is described in Lonberg and Huszar, Int. Rev. Immunol. 13:65 (1995). Transgenic animals with one or more human immunoglobulin genes (kappa or lambda) that do not express endogenous immunoglobulins are described, for example in, U.S. Pat. No. 5,939,598. Additional methods for producing human antibodies and human monoclonal antibodies are described (see, e.g., WO 98/24893; WO 92/01047; WO 96/34096; WO 96/33735; U.S. Pat. Nos. 5,413,923; 5,625,126; 5,633,425; 5,569,825; 5,661,016; 5,545,806; 5,814,318; 5,885,793; 5,916,771; and 5,939,598).

Antibodies can be humanized using a variety of techniques known in the art including, for example, CDR-grafting (EP 239,400; WO91/09967; U.S. Pat. Nos. 5,225,539; 5,530,101; and 5,585,089), veneering or resurfacing (EP 592,106; EP 519,596; Padlan, Molecular Immunol. 28:489 (1991); Studnicka et al., Protein Engineering 7:805 (1994); Roguska. et al., Proc. Nat'l. Acad. Sci. USA 91:969 (1994)), and chain shuffling (U.S. Pat. No. 5,565,332). Human consensus sequences (Padlan Mol. Immunol. 31:169 (1994); and Padlan Mol. Immunol. 28:489 (1991)) have previously used to humanize antibodies (Carter et al. Proc. Natl. Acad. Sci. USA 89:4285 (1992); and Presta et al. J. Immunol. 151:2623 (1993)).

Methods for producing chimeric antibodies are known in the art (e.g., Morrison, Science 229:1202 (1985); Oi et al., BioTechniques 4:214 (1986); Gillies et al., (1989) J. Immunol. Methods 125:191; and U.S. Pat. Nos. 5,807,715; 4,816,567; and 4,816,397). Chimeric antibodies in which a variable domain from an antibody of one species is substituted for the variable domain of another species are described, for example, in Munro, Nature 312:597 (1984); Neuberger et al., Nature 312:604 (1984); Sharon et al., Nature 309:364 (1984); Morrison et al., Proc. Nat'l. Acad. Sci. USA 81:6851 (1984); Boulianne et al., Nature 312:643 (1984); Capon et al., Nature 337:525 (1989); and Traunecker et al., Nature 339:68 (1989).

In some embodiments, an antibody may include recombinant antibody molecules, or fragments thereof, expressed from cloned antibody-encoding polynucleotides, such as polynucleotides isolated from hybridoma cells or selected from libraries of naturally occurring or synthetic antibody genes (see for example, Gram et al., Proc. Natl. Acad. Sci. USA 89:3576-80 (1992)).

In some embodiments, a composition may comprise a plurality of active agents. For example, a composition may include a first active agent that is a compound capable of binding galectin-3 and a second active agent (e.g., an active pharmaceutical ingredient). In some embodiments, a compound capable of binding galectin-3 may be combined with an active agent (e.g., an active pharmaceutical ingredient) suitable for the treatment of heart failure. Non-limiting examples of active agents include angiotensin-converting enzyme (ACE) inhibitors, antiplatelet agents, angiotensin II receptor blockers, beta blockers, calcium channel blockers, diuretics, vasodilators, digitalis preparations, and statins.

In some embodiments, an inhibitor of galectin-3 may have a minimum inhibitory concentration of less than about 1 mg/mL, less than about 500 micrograms/mL, less than about 200 micrograms/mL, less than about 100 micrograms/mL, less than about 50 micrograms/mL, less than about 20 micrograms/mL, less than about 10 micrograms/mL, less than about 5 micrograms/mL, or less than about 1 microgram/mL. In some cases, an inhibitor of galectin-3 may have a minimum inhibitory concentration between about 1 microgram/mL and about 1 mg/mL, between about 1 microgram/mL and about 500 micrograms/mL, between about 1 microgram/mL and about 100 micrograms/mL, between about 5 micrograms/mL and about 500 micrograms/mL, between about 5 microgram/mL and about 100 micrograms/mL, between about 1 microgram/mL and about 50 micrograms/mL, or between about 1 microgram/mL and about 10 micrograms/mL. Inhibitors may be identified, for example, by screening compounds suspected of having galectin-3 binding properties. For example, affinity chromatography using a chromatography resin comprising galectin-3 may be used to capture compounds displaying galectin-3 binding activity. Subsequently, liquid chromatography and mass spectrometry may be used to identify the compounds captured during the affinity chromatography step. One of ordinary skill in the art would readily contemplate other methods and assays for screening compounds suspected of having galectin-3 binding properties.

In some instances, a compound capable of binding to galectin-3 may be selected based on a desired pharmacological half-life. For example, in some embodiments, a compound capable of binding to galectin-3 may have a pharmacological half-life of between about 0.5 hours and about 2 hours, between about 1 hour and about 4 hours, between about 2 hours and about 6 hours, between about 4 hours and about 8 hours, between about 6 hours and about 10 hours, between about 8 hours and about 12 hours, or between about 0.5 hours and about 12 hours. In some embodiments, a compound capable of binding to galectin-3 may be modified so as to modulate the pharmacological half-life.

In some embodiments, binding of a compound to galectin-3 may inhibit an activity of galectin-3. For instance, binding of a compound to galectin-3 may inhibit an interaction between galectin-3 and a biological target, for example, a protein-protein interaction between galectin-3 and another protein, such as a receptor. As discussed above, galectin-3 has been shown to play a role in a variety of cellular process, including cell-cell adhesion, cell-matrix interactions, phagocytosis, cell cycle, apoptosis, angiogenesis, and mRNA splicing. In some cases, inhibition of galectin-3 may inhibit one or more of these processes. However, it should be understood that inhibition of galectin-3 may inhibit other processes as well including inflammation, fibrosis, activation of fibroblasts, organ remodeling, and the like. As galectin-3 has been shown to function through both intracellular and extracellular actions, binding of a compound to galectin-3 may inhibit an intracellular action, an extracellular action, or both an intracellular action and an extracellular action.

In one aspect, inhibition of galectin-3 may be used to treat a condition, such as a disease. In some embodiments, inhibition of galectin-3 may be used to treat conditions of the heart. For example, heart failure, cardiovascular disease, myocardial infarction, cancer, inflammatory diseases, and immunological disease are contemplated for treatment. This list is not meant in any way to be limiting, and other diseases and conditions may be treated as well. In some embodiments, inhibition of galectin-3 may be used to reduce the risk of developing a disease or condition (e.g., heart failure).

In some embodiments, binding of a compound to galectin-3 may inhibit an activity of galectin-3 relative to galectin-3 that is not bound to the compound. In some instances, the activity may be the carbohydrate-binding activity of galectin-3. In some cases, an activity of galectin-3 may be assayed by quantifying a marker of a galectin-3 activity. For example, a cell line having elevated levels of galectin-3 may be contacted with a compound capable of binding galectin-3. In one example, a change in an activity such as apoptosis may be assayed to determine if the compound inhibits an apoptosis-inducing activity of galectin-3. In some embodiments, a compound capable of binding galectin-3 may reduce the expression level of galectin-3. The expression level of galectin-3 may be determined using any of a variety of methods known in the art, such as ELISA or Western blotting.

In some cases, a compound capable of binding galectin-3 may inhibit an activity of galectin-3 by at least about 5%, at least about 10%, at least about 15%, at least about 20%, at least about 30%, at least about 40%, at least about 50%, at least about 60%, at least about 70%, at least about 80%, or at least about 90%.

In some embodiments, a compound capable of binding galectin-3 may reduce the expression level of galectin-3 by at least about 5%, at least about 10%, at least about 15%, at least about 20%, at least about 30%, at least about 40%, at least about 50%, at least about 60%, at least about 70%, at least about 80%, or at least about 90%.

### Methods

In another aspect, methods of treating a subject having heart failure or at risk of developing heart failure are contemplated. In some embodiments, a composition comprising a compound capable of binding to galectin-3 may be administered to a subject and may at least partially alleviate a symptom of heart failure. For example, in certain embodiments, cardiac fibrosis may be at least partially inhibited. In another embodiment, fractional shortening may be at least partially inhibited from decreasing or may be increased. In some embodiments, left ventricular ejection fraction may be at least partially inhibited from decreasing or may be increased. In other embodiments, right ventricular end diastolic pressure (RVEDP) may be at least partially inhibited from increasing or may be reduced. In yet another embodiment, left ventricle end diastolic pressure (LVEDP) may be at least partially inhibited from increasing or may be reduced. In some embodiments, left ventricular end diastolic volume may be at least partially inhibited from decreasing or may be increased. In other embodiments, left ventricular end systolic volume may be at least partially inhibited from decreasing or may be increased.

In still another embodiment, left ventricle relaxation constant (Tau) may be inhibited from increasing or may be reduced. In another embodiment, cardiac remodeling may be inhibited. In some embodiments, alleviating a symptom may refer to a reduction in the frequency of occurrence of a symptom. In other embodiments, alleviating a symptom may refer to a slowing of the development of a symptom. For example, cardiac fibrosis may occur over a period of time, and treating a subject with compound capable of binding to galectin-3 may slow the progress of cardiac fibrosis.

In some embodiments, fractional shortening and/or left ventricular ejection fraction and/or left ventricular end diastolic volume and/or left ventricular end systolic volume may be increased by at least about 5% or at least about 10%. In some embodiments, LVEDP and/or RVEDP may be decreased by at least about 1 mmHg, at least about 2 mmHg, at least about 3 mmHg, at least about 4 mmHg, or at least about 5 mmHg. In certain embodiments, Tau may be reduced by at least about 1 msec, about 2 msec, about 3 msec, about 4 msec, or about 5 msec.

In some instances, the rate of progression of a symptom of heart failure may be slowed by at least about 5%, at least about 10%, at least about 20%, at least about 30%, at least about 40%, at least about 50%, at least about 60%, at least about 70%, at least about 80%, or at least about 90% when a subject is treated with a compound capable of binding to galectin-3.

In some cases, a compound capable of binding to galectin-3 may reduce the risk of a subject developing heart failure. In some embodiments, the risk may be reduced by at least about 5%, at least about 10%, at least about 15%, at least about 20%, at least about 30%, at least about 40%, at least about 50%, at least about 60%, at least about 70%, at least about 80%, at least about 90%, at least about 95%, at least about 99% as compared to an untreated subject. In some embodiments, treating a subject at risk of developing heart failure may reduce the subject's risk of developing heart failure to that of normal risk.

In some embodiments, administering a compound capable of binding to galectin-3 to a subject may increase the 1 year survival rate, the 2 year survival rate, the 5-year survival rate, or the 10 year survival rate of the subject. In some instances, the survival rate may increase by at least about 5%, at least about 10%, at least about 15%, at least about 20%, at least about 30%, at least about 40%, at least about 50%, at least about 60%, at least about 70%, at least about 80%, at least about 90%, at least about 100%, at least about 200%, or at least about 300%.

In some embodiments, the activity of galectin-3 in a subject (e.g., a patient) may be determined. For instance, the activity of galectin-3 in a subject may be determined as part of a therapeutic regimen. For example, a composition comprising a compound capable of binding to galectin-3 may be administered to a subject (e.g., a patient) and the activity of galectin-3 in the subject may be determined. Such a regimen may be advantageous, for instance, for determining properties such as the proper dosage of the composition, the pharmacokinetics of the composition, the efficacy of the composition, and the like. An activity of galectin-3 may be determined by any method described herein or known to one of ordinary skill in the art. In some embodiments, determining the activity of galectin-3 may comprise determining the fraction of galectin-3 in a biological sample bound to the compound. For instance, an antibody assay may be used where the antibody binds to unbound galectin-3 but does not bind to galectin-3 bound to an inhibitor.

In some cases, a subject (e.g., a patient) may be identified for treatment by the level of galectin-3 in the subject. For example, a subject may be identified on the basis that the subject has elevated circulatory levels of galectin-3 as compared to a healthy subject. Methods and kits for determining the level of galectin-3 in a subject are disclosed in U.S. patent application Ser. No. 12/608,821, by Muntendam et al., filed on Oct. 29, 2009, and are described in more detail below. In some embodiments, determining the levels of galectin-3 in a subject may comprise obtaining a biological sample (e.g., blood, urine, tissue, and the like) from the subject. In some embodiments, a subject may be identified on the basis that the circulatory levels of galectin-3 are at least about 15 ng/mL, at least about 20 ng/mL or at least about 30 ng/mL. In some embodiments, a subject may be identified on the basis that the circulatory levels of galectin-3 are between at about 15 ng/mL and about 20 ng/mL, between about 20 ng/mL and about 25 ng/mL, between about 25 ng/mL and about 30 ng/mL, or between about 30 ng/mL and about 35 ng/mL. In some embodiments, a subject may be identified on the basis that the circulatory levels of galectin-3 are at least about 10% elevated as compared to a standard level, at least about 20% elevated as compared to a standard level, or at least about 50% elevated as compared to a standard level.

In another aspect, a method of predicting and/or monitoring a heart failure patient's physiological response to treatment of heart failure with an aldosterone antagonist provided. Without wishing to be bound by any theory, it is believed that aldosterone antagonists are diuretic drugs which antagonize the action of aldosterone at mineralocorticoid receptors. Currently-available aldosterone antagonists include spironolactone, eplerenone, canrenone, prorenone, and mexrenone. Without wishing to be bound by any theory, it is believed that aldosterone induces galectin-3.

In some embodiments, circulating galectin-3 protein levels may be used to predict efficacy of aldosterone antagonist treatments in heart failure patients. Patients whose galectin-3 levels identify them as candidates for aldosterone antagonist treatment can be treated by repeated administration of a aldosterone antagonist.

In some cases, treatment with an aldosterone antagonist may be optionally combined with one or more other treatments for heart failure. For example, a patient may also be treated with a compound that is capable of binding to galectin-3, as described elsewhere here, a diuretic, such as furosemide, bumetanide, hydrochlorothiazide, spironolactone, eplerenone, triamterene, torsemide, or metolazone; an inotrope, such as dobutamine, milrinone, or digoxin; a beta-blocker, such as carvediol or metoprolol; and/or a natriuretic peptide, such as BNP. In some embodiments, treatments can also include a vasodilator, such as: an angiotensin-converting enzyme (ACE) inhibitor (e.g. captopril, enalapril, lisinopril, benazepril, quinapril, fosinopril, or ramipril); an angiotensin II receptor blocker, such as candesartan, irbesartan, olmesartan, losartan, valsartan, telmisartan, or eprosartan; a nitrate, such as isosorbide mononitrate or isosorbide dinitrate; and/or hydralazine. Other forms of medical intervention, such as angioplasty, implantation of a pacemaker, or other surgery can also be performed in appropriate cases.

In certain embodiments, galectin-3 levels and/or other biomarkers (such as BNP) can be measured in a patient taking a galectin-3 inhibitor and/or aldosterone antagonist and can be compared to a previous galectin-3 concentration measured in the patient. In some instances, an increase or decrease in galectin-3 concentration relative to one or more previous galectin-3 concentrations in the patient may be an indication that the patient is responding or not responding to galectin-3 inhibitor and/or aldosterone antagonist therapy. Marker levels can be monitored over time, such as in samples obtained from a patient at annual, semi-annual, bimonthly, monthly, triweekly, biweekly, weekly, daily, or at variable intervals.

In some cases, treatment with an aldosterone antagonist may be modified if the patient is determined to be not responding to aldosterone antagonist thereapy. For example, the aldosterone antagonist dosage amount may be increased or the frequency of administration may be increased until the patient's level of galectin-3 is reduced to an acceptable level.

In another aspect, the methods described herein may be used to determine a galectin-3 inhibitory activity of a foodstuff. In some embodiments, a foodstuff may be administered to a subject and the activity of galectin-3 in the subject after administration of the foodstuff may be determined. In some instances, the subject may have elevated circulatory levels of galectin-3 (e.g., prior to administration of the foodstuff). In some embodiments, a subject may have circulatory levels of galectin-3 that are at least about 15 ng/mL. In certain embodiments, the activity of galectin-3 in a subject may be compared to a standard activity. For example, a subject may have circulatory levels of galectin-3 are at least about 10% elevated as compared to a standard level, at least about 20% elevated as compared to a standard level, or at least about 50% elevated as compared to a standard level. In some embodiments, the activity of galectin-3 in the subject may be determined prior to administration of the foodstuff. In certain embodiments, the activity of galectin-3 in the subject prior to administration of the foodstuff may be compared to the activity of galectin-3 in the subject after administration of the foodstuff.

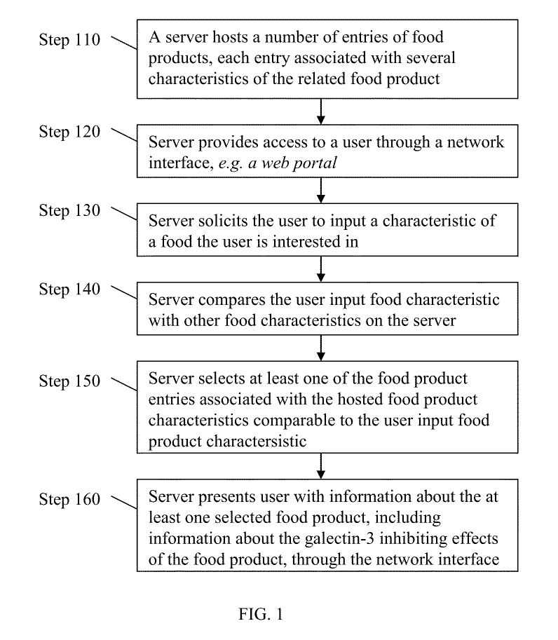

Users may wish to access information relating to the galectin-3 inhibiting effects of various food products (e.g., foodstuffs), including an assigned rating. This is depicted in the flowchart in FIG. 1. To facilitate this presentation of information, a server may be provided, as described in greater detail below. The server may host a number of entries relating to various food products (Step 110). Each food product (e.g., foodstuff) may have a number of characteristics associated with it, such as the name of the food, the food group the food belongs to, the caloric content of the food, and the galectin-3 inhibiting rating, amongst others. These characteristics form a database that may be accessed by a user through a network interface, such as through a web portal (Step 120).

In one embodiment, a user may access a website connected to the server. The website may present the user with an option to input a characteristic of the food they are interested in (Step 130). This may be, but is not limited to, the name of the food, e.g., “carrot.” The server may locate food entries with similar characteristics as the user input food characteristic (Step 140). In this example, the server may locate any foods with name characteristics similar to “carrot,” and then select at least one of these foods (Step 150) to display information related thereto, including information relating to the galectin-3 inhibiting effects of the food, to the user (Step 160). In some embodiments, several different food products may be presented, such as, based on the example, carrot, carrot cake, and carrot juice. Different preparations of the same food may also be presented, such as raw and cooked. The information relating to each of these may be displayed simultaneously, or may only be displayed once a particular food product is selected by the user. The server may also be designed to present alternative choices for the user. For example, the server may find other foods with characteristics similar to those of carrots, such as other vegetables and/or foods that are in a similar caloric range. These alternative options may be presented to the user and accessed as described above. The alternative options may be anything with similar qualities, or may be limited to foods with a higher galectin-3 inhibiting rating. Other changes in the selection and presentation are contemplated, such as displaying other foods that may substitute for the user input food (e.g., when the food is being used as an ingredient). As can be appreciated, the information presented to the user may range from a single entry to several entries, and may be customizable by the user themselves. Additionally, the user may input multiple foods at once.

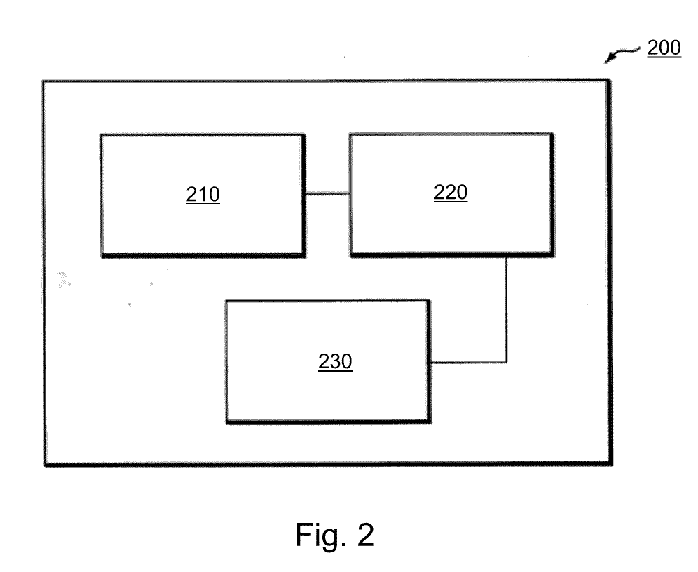

A hosting server 200 for use with one aspect of the invention is schematically depicted in FIG. 2. The server 200 includes a network interface 210, a processor 220, and a database 230. The server 200 hosts a number of the food product characteristics described above. These food product characteristics may be hosted in the database 230, which can be accessed by other network connected sources through the network interface 210 and the processor 220. The database 230 may be organized based on the properties of food product characteristics, and may update automatically or through manual additions. Certain content (such as the food product information) on the server 230 may be accessible to a large population, while other content (such as confidential documents) may be restricted to a limited population.

### Detection of Galectin-3 by Sandwich Assay

In some embodiments, the concentration of galectin-3 may be quantitated in a bodily fluid sample using a pair of binding moieties that bind specifically to N-terminal portions of galectin-3. A “binding moiety” refers to a molecule that binds or interacts selectively or preferentially with a polypeptide or peptide. Examples of binding moieties include, but are not limited to, proteins, such as antibodies, galectin binding protein (GBP) interaction fusion protein, peptide aptamers, avimers, Fabs, sFvs, Adnectins and Affibody® ligands; nucleic acids, such as DNA and RNA (including nucleotide aptamers), and lipids, such as membrane lipids.

In certain embodiments, detection of the concentration of galectin-3 in a clinical sample, such as serum, for the diagnosis of HF may be made. The test sample used in the detection of galectin-3 can be any body fluid or tissue sample, including, but not limited to, whole blood, serum, plasma, or lymph, and less preferably urine, gastric juices, bile, saliva, sweat, and spinal fluids, stool, or muscle biopsy. In a preferred embodiment, the sample is a blood sample. In another embodiment, the sample is a plasma sample. Serum samples may also be used. Furthermore, the body fluids may be either processed (e.g., serum) or unprocessed. Methods of obtaining a body fluid from a subject are known to those skilled in the art.

In some embodiments, galectin-3 may be detected and quantified using a “sandwich” assay. In this embodiment, two molecules (“binding moieties”) such as monoclonal antibodies that specifically bind to non-overlapping sites (“epitopes”) on the N-terminus of galectin-3 are used. Typically, one binding moiety is immobilized on a solid surface where it binds with and captures galectin-3. This first binding moiety is therefore also referred to herein as the capture binding moiety. A second binding moiety is detectably labeled, for example, with a fluorophore, enzyme, or colored particle, such that binding of the second binding moiety to the galectin-3-complex indicates that galectin-3 has been captured. The intensity of the signal is proportional to the concentration of galectin-3 in the sample. The second binding moiety is therefore also referred to herein as the detection binding moiety or label binding moiety. A binding moiety can be any type of molecule, as long as it specifically binds to a portion of the N-terminus of galectin-3. In a preferred embodiment, the binding moieties used are monoclonal anti-galectin-3 antibodies, i.e., monoclonals raised against or otherwise selected to bind to separate portions of the N-terminal 113 amino acids of galectin-3.

Such assay procedures can be referred to as two-site immunometric assay methods, “sandwich” methods or (when antibodies are the binders) “sandwich immunoassays.” As is known in the art, the capture and detection antibodies can be contacted with the test sample simultaneously or sequentially. Sequential methods, sometimes referred to as the “forward” method, can be accomplished by incubating the capture antibody with the sample, and adding the labeled detection antibody at a predetermined time thereafter. Alternatively, the labeled detection antibody can be incubated with the sample first and then the sample can be exposed to the capture antibody (sometimes referred to as the “reverse” method). After any necessary incubation(s), which may be of short duration, the label is detected and may also be measured. Such assays may be implemented in many specific formats known to those of skill in the art, including through use of various high throughput clinical laboratory analyzers or with point of care or home testing devices.

In one embodiment, a lateral flow device may be used in the sandwich format, wherein the presence of galectin-3 above a baseline sensitivity level in a biological sample will permit formation of a sandwich interaction upstream of or at the capture zone in the lateral flow assay. See, for example, U.S. Pat. No. 6,485,982. The capture zone as used herein may contain capture binding moieties such as antibody molecules, suitable for capturing galectin-3, or immobilized avidin or the like for capture of a biotinylated complex. See, for example, U.S. Pat. No. 6,319,676. The device may also incorporate a luminescent label suitable for capture in the capture zone, the concentration of galectin 3 being proportional to the intensity of the signal at the capture site. Suitable labels include fluorescent labels immobilized on polystyrene microspheres. Colored particles also may be used.

Other assay formats that may be used in the methods of the invention include, but are not limited to, flow-through devices. See, for example, U.S. Pat. No. 4,632,901. In a flow-through assay, one binding moiety (for example, an antibody) is immobilized to a defined area on a membrane surface. This membrane is then overlaid on an absorbent layer that acts as a reservoir to pump sample volume through the device. Following immobilization, the remaining protein-binding sites on the membrane are blocked to minimize non-specific interactions. In operation, a biological sample is added to the membrane and filters through, allowing any analyte specific to the antibody in the sample to bind to the immobilized antibody. In a second step, a labeled secondary antibody may be added or released that reacts with captured marker to complete the sandwich. Alternatively, the secondary antibody can be mixed with the sample and added in a single step. If galectin-3 is present, a colored spot develops on the surface of the membrane.

The most common enzyme immunoassay is the “Enzyme-Linked Immunosorbent Assay (ELISA).” ELISA is a technique for detecting and measuring the concentration of an antigen using a labeled (e.g., enzyme linked) form of the antibody. There are different forms of ELISA, which are well known to those skilled in the art. The standard techniques known in the art for ELISA are described in “Methods in Immunodiagnosis”, 2nd Edition, Rose and Bigazzi, eds. John Wiley & Sons, 1980; Campbell et al., “Methods and Immunology”, W. A. Benjamin, Inc., 1964; and Oellerich, M. (1984), J. Clin. Chem. Clin. Biochem. 22:895-904.

In a “sandwich ELISA,” an antibody (e.g., anti-galectin-3) is linked to a solid phase (i.e., a microtiter plate) and exposed to a biological sample containing antigen (e.g., galectin-3). The solid phase is then washed to remove unbound antigen. A labeled antibody (e.g., enzyme linked) is then bound to the bound antigen, forming an antibody-antigen-antibody sandwich. Examples of enzymes that can be linked to the antibody are alkaline phosphatase, horseradish peroxidase, luciferase, urease, and β-galactosidase. The enzyme-linked antibody reacts with a substrate to generate a colored reaction product that can be measured. This measurement can be used to derive the concentration of galectin-3 present in a sample, for example, by comparing the measurement to a galectin-3 standard curve. Galectin-3 concentration (e.g., blood concentration) in a sample from a subject may be determined to be above or below a threshold or within a target range. The threshold may be in the range of, for example, about 5-10 ng/ml, about 0-15 ng/ml; about 15-20 ng/ml; about 20-25 ng/ml; about 25-30 ng/ml; about 30-35 ng/ml, or about 35-40 ng/ml. In some instances, the minimum threshold may be more than 10 ng/ml. In some embodiments, the minimum threshold may be more than 30 ng/ml. In some cases, the galectin-3 blood concentration may be determined to be below a maximum threshold. For example, the maximum threshold may be below about 70 ng/ml, below about 60 ng/ml, or below about 40 ng/ml. The maximum threshold may be between about 30 and about 40 ng/ml, between about 25 and about 30 ng/ml, between about 20 and about 25 ng/ml, or between about 15 and about 20 ng/ml.

Any of the immunoassays described herein suitable for use with the kits and methods can also use any binding moiety in the place of an antibody.

### Binding Moieties

In a preferred embodiment of the invention, anti-galectin-3 antibodies, preferably monoclonal antibodies, are used as binding moieties. However, it should also be understood that the binding moieties described below may also be administered as galectin-3 inhibitors.

### Monoclonal Antibodies

In preferred embodiments of the invention, monoclonal antibodies are used. A monoclonal antibody refers to an antibody that is derived from a single clone, including any eukaryotic, prokaryotic, or phage clone. The monoclonal antibody may comprise, or consist of, two proteins, i.e., heavy and light chains. The monoclonal antibody can be prepared using one of a wide variety of techniques known in the art including the use of hybridoma, recombinant, and phage display technologies, or a combination thereof.

Anti-galectin-3 monoclonal antibodies may be prepared using any known methodology, including the seminal hybridoma methods, such as those described by Kohler and Milstein (1975), Nature. 256:495. In a hybridoma method, a mouse, hamster, or other appropriate host animal is immunized with an immunizing agent to elicit lymphocytes that produce or are capable of producing antibodies that will specifically bind to the immunizing agent. Alternatively, the lymphocytes may be immunized in vitro.

The immunizing agent will typically include at least a portion of the galectin-3 polypeptide or a fusion protein thereof. For example, synthetic polypeptide or recombinant polypeptide comprising any galectin-3 N-terminal epitopes may be used as an immunizing agent. Exemplary N-terminal epitopes include, but are not limited to, MADNFSLHDALS (SEQ ID NO:1), MADNFSLHDALSGS (SEQ ID NO:2), GNPNPQGWPGA (SEQ ID NO:3), WGNQPAGAGG (SEQ ID NO:4), YPGQAPPGAYPGQAPPGA (SEQ ID NO:5), YPGAPGAYPGAPAPGV (SEQ ID NO:6), YPGAPAPGVYPGPPSGPGA (SEQ ID NO:7), YPSSGQPSATGA (SEQ ID NO:8). A fusion protein may be made by fusing a polypeptide to a carrier protein, for example, keyhole limpet hemocyanin (KLH, EMD Biosciences, San Diego, Calif.), BSA (EMD Biosciences, San Diego, Calif.), or ovalbumin (Pierce, Rockford, Ill.). The immunizing agent may be administered to a mammal with or without adjuvant according to any of a variety of standard methods. The immunizing agent may be administered only once, but is preferably administered more than once according to standard boosting schedules.

Generally, either peripheral blood lymphocytes (“PBLs”) are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell population which is screened for species having appropriate specificity and affinity to epitopes on the N-terminal portion of galectin-3 (Goding, (1986) Monoclonal Antibodies: Principles and Practice, Academic Press, pp. 59-103). Immortalized cell lines are usually transformed mammalian cells, particularly myeloma cells of rodent, bovine and human origin. Usually, rat or mouse myeloma cell lines are employed. The hybridoma cells may be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

Preferred immortalized cell lines are those that fuse efficiently, support stable high level expression of antibody by the selected antibody-producing cells, and are sensitive to a medium such as HAT medium. More preferred immortalized cell lines are murine myeloma lines, which can be obtained, for instance, from the Salk Institute Cell Distribution Center, San Diego, Calif. and the American Type Culture Collection, Manassas, Va. Human myeloma and mouse-human heteromyeloma cell lines also have been described for the production of human monoclonal antibodies (Kozbor, J. (1984) Immunol., 133:3001; Brodeur et al., Monoclonal Antibody Production Techniques and Applications, Marcel Dekker, Inc., New York, (1987) pp. 51-63).

The culture medium in which the hybridoma cells are cultured can then be assayed for the presence of monoclonal antibodies directed against the N-terminus of galectin-3, e.g., by screening with a labeled galectin-3 N-terminal polypeptide. Preferably, the binding specificity of monoclonal antibodies produced by the hybridoma cells is determined by immunoprecipitation or by an in vitro binding assay, such as radioimmunoassay (RIA) or enzyme-linked immunoabsorbent assay (ELISA). Such techniques and assays are known in the art. The binding affinity of the monoclonal antibody can, for example, be determined by the Scatchard analysis of Munson and Pollard (1980), Anal. Biochem., 107:220. Various analysis protocols to determine binding specificity are available commercially as kits or as a service.

Monoclonal antibodies also may be made by recombinant DNA methods, such as those described in U.S. Pat. No. 4,816,567. DNA encoding suitable monoclonal antibodies can be isolated and sequenced using conventional procedures (e.g., by using oligonucleotide probes that are capable of binding specifically to genes encoding the heavy and light chains of murine antibodies). The hybridoma cells serve as a preferred source of such DNA. Once isolated, the DNA may be placed into expression vectors, which are then transfected into host cells such as simian COS cells, Chinese hamster ovary (CHO) cells, or myeloma cells that do not otherwise produce immunoglobulin protein, to obtain the synthesis of monoclonal antibodies in the recombinant host cells. The DNA also may be modified, for example, by substituting the coding sequence for human heavy and light chain constant domains in place of the homologous murine sequences (U.S. Pat. No. 4,816,567; Morrison et al., (1984) Proc. Natl. Acad. Sci. USA, 81:6851) or by covalently joining to the immunoglobulin coding sequence all or part of the coding sequence for a non-immunoglobulin polypeptide. Such a non-immunoglobulin polypeptide can be substituted for the constant domains of an antibody of the invention, or can be substituted for the variable domains of one antigen-combining site of an antibody of the invention to create a chimeric bivalent antibody.

The antibodies may be monovalent antibodies. Methods for preparing monovalent antibodies are well known in the art. For example, one method involves recombinant expression of immunoglobulin light chain and modified heavy chain. The heavy chain is truncated generally at any point in the Fc region so as to prevent heavy chain crosslinking Alternatively, the relevant cysteine residues are substituted with another amino acid residue or are deleted so as to prevent crosslinking.

In vitro methods are also suitable for preparing monovalent antibodies. Digestion of antibodies to produce fragments thereof, particularly, Fab fragments, can be accomplished using routine techniques known in the art.

Antibodies can also be produced using phage display libraries (Hoogenboom and Winter (1991), J. Mol. Biol. 227:381; Marks et al. (1991), J. Mol. Biol., 222:581). The techniques of Cole et al. and Boerner et al. are also available for the preparation of monoclonal antibodies (Cole et al. (1985), Monoclonal Antibodies and Cancer Therapy, Alan R. Liss, p. 77 and Boerner et al. (1991), J. Immunol., 147(1):86-95). Similarly, antibodies can be made by introducing of immunoglobulin loci into transgenic animals, e.g., mice in which the endogenous immunoglobulin genes have been partially or completely inactivated.

The antibodies may also be affinity matured using known selection and/or mutagenesis methods as described above. Preferred affinity matured antibodies have an affinity which is five times, more preferably 10 times, even more preferably 20 or 30 times greater than the starting antibody from which the matured antibody is prepared. In a particularly preferred embodiment, the antibodies used to detect galectin-3 are monoclonal antibodies, for example, M3/38, 9H3.2, and 87B5. M3/38 detects a linear epitope (YPGQAPPGAYPGQAPPGA (SEQ ID NO:5)) on the N-terminus of galectin-3. M3/38 was prepared from the supernatant of the rat hybridoma M3/38.1.2.8 HL.2, a clone of which can be found in the American Type Culture Collection with ATCC® number TIB-166. 9H3.2 detects a linear epitope (MADNFSLHDALSGS (SEQ ID NO:2) at the extreme N-terminus of galectin-3. 9H3.2 is a mouse monoclonal IgG, affinity purified using protein A. 9H3.2 is available from Millipore (Millipore, 290 Concord Road, Billerica, Mass. 01821, USA), catalog no.: MAB4033. 87B5 detects a non-linear epitope comprising portions of GNPNPQGWPGA (SEQ ID NO:3) and YPGAPAPGVYPGPPSGPGAYPSSGQPSATGA (SEQ ID NO:9). 87B5 was prepared from the mouse-mouse hybridoma (X63-Ag8.653xBALB/c mouse spleen cells) clone 87B5, and is an IgG2a that was affinity purified using Protein A. 87B5 is available from Immuno-Biological Laboratories (IBL, 8201 Central Ave NE, Suite P, Minneapolis, Minn. 55432 USA).

In a currently preferred embodiment, the capture binding moiety is the anti-galectin-3 monoclonal antibody, M3/38 and the labeled detection binding moiety is a second anti-galectin-3 monoclonal antibody, 87B5. The given designations for these antibodies are not limiting. In another embodiment, the capture antibody is 9H3.2 and the labeled detection binding moiety is M3/38. Other antibodies which recognize the epitopes described above also may be used.

Other binding moieties may be used with the methods and kits of the present invention. Examples of binding moieties include, but are not limited to, proteins, peptide aptamers, avimers, Adnectins and Affibody® ligands; nucleic acids, such as DNA and RNA (including nucleotide aptamers), and lipids, such as membrane lipids.

### Aptamers

Nucleotide aptamers are small peptides or small nucleotide sequences that bind with high affinity to a target of choice. Nucleotide aptamers are produced by a selection process called Systematic Evolution of Ligands by Exponential Enrichment (SELEX), also referred to as in vitro selection or in vitro evolution. In this procedure, a target molecule is exposed to a large, randomly generated oligonucleotide library. The unbound oligonucleotides are separated out of the mixture by any number of methods, usually affinity chromatography. The oligonucleotides that remain bound are eluted and amplified. The target molecule is then exposed to the newly-synthesized oligonucleotides, and the selection process is repeated for several rounds with increasingly stringent conditions to separate out unbound sequences. The resulting oligonucleotides are then sequenced to determine their identity. See U.S. patent application Ser. No. 07/536,428; U.S. Pat. No. 5,475,096; and U.S. Pat. No. 5,270,163.

Peptide aptamers typically consist of a short variable peptide domain. Peptide aptamers comprise a variable peptide loop attached at both ends to a protein scaffold. This double structural constraint greatly increases the binding affinity of the peptide aptamer to levels comparable to an antibody's (nanomolar range). The variable loop length is typically 10 to 20 amino acids, and the scaffold may be any protein that is soluble and compact, such as the bacterial protein thioredoxin A. A variable loop can be inserted within the reducing active site of thioredoxin A, which is a -Cys-Gly-Pro-Cys- (SEQ ID NO:10) loop in the wild protein, the two cysteine lateral chains being able to form a disulfide bridge. Peptide aptamer selection can be made using different systems, such as the yeast two-hybrid system. For further discussion of peptide aptamers, see International Patent Publication No. WO2007/117657.

### Avimers

An avimer (avidity multimer) is a short peptide sequence that contains multiple regions of low affinity to a target. The presence of multiple unique regions of low affinity act together to produce a high affinity binding moiety. Small size and high disulfide density contribute to the low immunogenicity of avimers. To identify avimers with high binding affinity for a protein of interest, a highly diverse pool of monomers is created by synthetic recombination. This pool of monomers can be screened against a target protein using phage display or another preferred screening method. Once candidates are found, another monomer is added and the new library of dimers is screened against the target. After iteration, a trimer with very high binding affinity for its target protein is isolated (see Silverman et al. (2005), Nature Biotechnology 23, 1556-1561).

### Adnectins

An Adnectin consists of a backbone of the natural amino acid sequence of a certain domain of human fibronectin and one to three targeting loops which contain randomized sequence. Adnectins are screened and isolated based on ability to specifically recognize a therapeutic target of interest (see, for example, U.S. Pat. No. 6,818,418).

### Affibody® Ligands

Affibody ligands are small peptides comprised of a “scaffold” domain and a variable domain. The “scaffold” domain comprises a non-cysteine three-helix bundle domain, a structure based on staphylococcal protein A. The variable domain contains randomly-generated sequences which can be screened against a target of interest. Libraries of Affibody® ligands are constructed, and the libraries can be screened to find candidates with high binding affinity to a protein of interest. See Nygren, P. A. (2008) FEBS Journal 275, 2668-2676.

### Naturally-Occurring Binding Partners

Naturally-occurring galectin-3 binding partners that bind to the N-terminus of galectin-3 can be isolated or produced recombinantly and used as binding moieties or therapeutic agents. Galectin-3 binding partners or fragments thereof can be used as either capture or detection binding moieties, depending upon the particular constraints of the assay. Examples of galectin-3 binding partners include, but are not limited to mycolic acids and lipopolysaccharides (Barboni et al. (2005) FEBS Letters 579:6749-6755). Additionally, circulating galectin-3 triggers an auto-immune response resulting in the generation of auto-antibodies against galectin-3 in serum under both normal and pathological conditions (Jensen-Jarolim et al. (2001) J Clin Immunol. 21(5):348-56; Lim et al., (2002) Biochem Biophys Res Commun. 295(1):119-24; Mathews et al. (1995) J Clin Immunol. 15(6):329-37). These auto-antibodies appear to target against epitopes on the N-terminal domain of galectin-3 (Mathews et al., supra).

### Alternate Forms of Galectin-3

Galectin-3 may exist in a sample in a plurality of different forms characterized by detectably different masses. These forms can result from pre-translational modifications, post-translational modifications or both. Pre-translational modified forms include allelic variants, splice variants, and RNA-editing forms. Post-translationally modified forms include forms resulting from, among other things, proteolytic cleavage (e.g., fragments of a parent protein), complexation, glycosylation, phosphorylation, lipidation, oxidation, methylation, cystinylation, sulphonation and acetylation. Modified forms of galectin-3, as long as they retain the relevant N-terminal epitopes, may be detected according to the methods of the present invention.

### Diagnostic and Prognostic Uses

A galectin-3 assay can be used to identify subjects at risk for developing or to identify subjects suffering from HF. In this method, patients or other subjects with an identified risk of developing HF may be monitored for changes in galectin-3 levels quantitated from a body fluid over time using an immunoassay of the invention. In certain embodiments, a subject with an identified risk to develop HF may monitor his or her galectin-3 levels over time, for example, monthly, quarterly, bi-yearly, yearly, every 2 years, or every 5 years.

In another embodiment, galectin-3 may be used as a diagnostic marker to determine the presence, stage or severity of HF in a subject or to predict his or her prognosis by measuring the concentration of galectin-3 in a sample and comparing this result to data correlating galectin-3 concentration with severity or stage of HF disease of human subjects. Methods of diagnosis and/or predicting prognosis described herein may be combined with other methods for diagnosis and/or predicting prognosis commonly used in the art, such as echocardiograms with Doppler analysis, radionuclide ventriculography, magnetic resonance imaging (MRI), a complete blood count, urinalysis, serum electrolytes, glycohemoglobin and blood lipids, tests of renal and hepatic function, tests of thyroid function, a chest radiograph, a 12-lead electrocardiogram, blood tests for biomarkers such as BNP, etc.

### Pharmaceutical Formulations

Disclosed compositions may be administered to patients (animals and humans) in need of such treatment in dosages that will provide optimal pharmaceutical efficacy. It will be appreciated that the dose required for use in any particular application will vary from patient to patient, not only with the particular compound or composition selected, but also with the route of administration, the nature of the condition being treated, the age and condition of the patient, concurrent medication or special diets then being followed by the patient, and other factors which those skilled in the art will recognize, with the appropriate dosage ultimately being at the discretion of the attendant physician. For treating clinical conditions and diseases noted above, a compound may be administered orally, subcutaneously, topically, parenterally, by inhalation spray or rectally in dosage unit formulations containing conventional non-toxic pharmaceutically acceptable carriers, adjuvants, and vehicles. Parenteral administration may include subcutaneous injections, intravenous or intramuscular injections, or infusion techniques.

Treatment can be continued for as long or as short a period as desired. The compositions may be administered on a regimen of, for example, one to four or more times per day. A suitable treatment period can be, for example, at least about one week, at least about two weeks, at least about one month, at least about six months, at least about 1 year, or indefinitely. A treatment period can terminate when a desired result, for example a partial or total alleviation of symptoms, is achieved.

In another aspect, pharmaceutical compositions comprising a compound capable of binding galectin-3 as disclosed herein formulated together with a pharmaceutically acceptable carrier are provided. In particular, the present disclosure provides pharmaceutical compositions comprising a compound capable of binding galectin-3 as disclosed herein formulated together with one or more pharmaceutically acceptable carriers. These formulations include those suitable for oral, rectal, topical, buccal, parenteral (e.g., subcutaneous, intramuscular, intradermal, or intravenous) rectal, vaginal, or aerosol administration, although the most suitable form of administration in any given case will depend on the degree and severity of the condition being treated and on the nature of the particular compound being used. For example, disclosed compositions may be formulated as a unit dose, and/or may be formulated for oral or subcutaneous administration.

Exemplary pharmaceutical compositions may be used in the form of a pharmaceutical preparation, for example, in solid, semisolid, or liquid form, which contains one or more of the compounds, as an active ingredient, in admixture with an organic or inorganic carrier or excipient suitable for external, enteral, or parenteral applications. The active ingredient may be compounded, for example, with the usual non-toxic, pharmaceutically acceptable carriers for tablets, pellets, capsules, suppositories, solutions, emulsions, suspensions, and any other form suitable for use. The active object compound is included in the pharmaceutical composition in an amount sufficient to produce the desired effect upon the process or condition of the disease.

For preparing solid compositions such as tablets, the principal active ingredient may be mixed with a pharmaceutical carrier, e.g., conventional tableting ingredients such as corn starch, lactose, sucrose, sorbitol, talc, stearic acid, magnesium stearate, dicalcium phosphate or gums, and other pharmaceutical diluents, e.g., water, to form a solid preformulation composition containing a homogeneous mixture of a compound, or a non-toxic pharmaceutically acceptable salt thereof. When referring to these preformulation compositions as homogeneous, it is meant that the active ingredient is dispersed evenly throughout the composition so that the composition may be readily subdivided into equally effective unit dosage forms such as tablets, pills and capsules.

In solid dosage forms for oral administration (capsules, tablets, pills, dragees, powders, granules and the like), the subject composition is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, acetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the subject composition moistened with an inert liquid diluent. Tablets, and other solid dosage forms, such as dragees, capsules, pills and granules, may optionally be scored or prepared with coatings and shells, such as enteric coatings and other coatings well known in the pharmaceutical-formulating art.

Compositions for inhalation or insufflation include solutions and suspensions in pharmaceutically acceptable, aqueous or organic solvents, or mixtures thereof, and powders. Liquid dosage forms for oral administration include pharmaceutically acceptable emulsions, microemulsions, solutions, suspensions, syrups and elixirs. In addition to the subject composition, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, cyclodextrins and mixtures thereof.

Suspensions, in addition to the subject composition, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations for rectal or vaginal administration may be presented as a suppository, which may be prepared by mixing a subject composition with one or more suitable non-irritating excipients or carriers comprising, for example, cocoa butter, polyethylene glycol, a suppository wax or a salicylate, and which is solid at room temperature, but liquid at body temperature and, therefore, will melt in the body cavity and release the active agent.

Dosage forms for transdermal administration of a subject composition includes powders, sprays, ointments, pastes, creams, lotions, gels, solutions, patches and inhalants. The active component may be mixed under sterile conditions with a pharmaceutically acceptable carrier, and with any preservatives, buffers, or propellants which may be required.

The ointments, pastes, creams and gels may contain, in addition to a subject composition, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays may contain, in addition to a subject composition, excipients such as lactose, talc, silicic acid, aluminum hydroxide, calcium silicates and polyamide powder, or mixtures of these substances. Sprays may additionally contain customary propellants, such as chlorofluorohydrocarbons and volatile unsubstituted hydrocarbons, such as butane and propane.

Compositions and compounds may alternatively be administered by aerosol. This is accomplished by preparing an aqueous aerosol, liposomal preparation or solid particles containing the compound. A non-aqueous (e.g., fluorocarbon propellant) suspension could be used. Sonic nebulizers may be used because they minimize exposing the agent to shear, which may result in degradation of the compounds contained in the subject compositions. Ordinarily, an aqueous aerosol is made by formulating an aqueous solution or suspension of a subject composition together with conventional pharmaceutically acceptable carriers and stabilizers. The carriers and stabilizers vary with the requirements of the particular subject composition, but typically include non-ionic surfactants (Tweens, Pluronics, or polyethylene glycol), innocuous proteins like serum albumin, sorbitan esters, oleic acid, lecithin, amino acids such as glycine, buffers, salts, sugars, or sugar alcohols. Aerosols generally are prepared from isotonic solutions.

Pharmaceutical compositions suitable for parenteral administration comprise a subject composition in combination with one or more pharmaceutically-acceptable sterile isotonic aqueous or non-aqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and non-aqueous carriers which may be employed in the pharmaceutical compositions include water, ethanol, polyols (such as glycerol, propylene glycol, polyethylene glycol, and the like), and suitable mixtures thereof, vegetable oils, such as olive oil, and injectable organic esters, such as ethyl oleate and cyclodextrins. Proper fluidity may be maintained, for example, by the use of coating materials, such as lecithin, by the maintenance of the required particle size in the case of dispersions, and by the use of surfactants.

In another aspect, enteral pharmaceutical formulations including a disclosed pharmaceutical composition comprising a compound capable of binding to galectin-3, an enteric material; and a pharmaceutically acceptable carrier or excipient thereof are provided. Enteric materials refer to polymers that are substantially insoluble in the acidic environment of the stomach, and that are predominantly soluble in intestinal fluids at specific pHs. The small intestine is the part of the gastrointestinal tract (gut) between the stomach and the large intestine, and includes the duodenum, jejunum, and ileum. The pH of the duodenum is about 5.5, the pH of the jejunum is about 6.5 and the pH of the distal ileum is about 7.5. Accordingly, enteric materials are not soluble, for example, until a pH of about 5.0, of about 5.2, of about 5.4, of about 5.6, of about 5.8, of about 6.0, of about 6.2, of about 6.4, of about 6.6, of about 6.8, of about 7.0, of about 7.2, of about 7.4, of about 7.6, of about 7.8, of about 8.0, of about 8.2, of about 8.4, of about 8.6, of about 8.8, of about 9.0, of about 9.2, of about 9.4, of about 9.6, of about 9.8, or of about 10.0. Exemplary enteric materials include cellulose acetate phthalate (CAP), hydroxypropyl methylcellulose phthalate (HPMCP), polyvinyl acetate phthalate (PVAP), hydroxypropyl methylcellulose acetate succinate (HPMCAS), cellulose acetate trimellitate, hydroxypropyl methylcellulose succinate, cellulose acetate succinate, cellulose acetate hexahydrophthalate, cellulose propionate phthalate, cellulose acetate maleate, cellulose acetate butyrate, cellulose acetate propionate, copolymer of methylmethacrylic acid and methyl methacrylate, copolymer of methyl acrylate, methylmethacrylate and methacrylic acid, copolymer of methylvinyl ether and maleic anhydride (Gantrez ES series), ethyl methyacrylate-methylmethacrylate-chlorotrimethylammonium ethyl acrylate copolymer, natural resins such as zein, shellac and copal collophorium, and several commercially available enteric dispersion systems (e.g., Eudragit L30D55, Eudragit FS30D, Eudragit L100, Eudragit S100, Kollicoat EMM30D, Estacryl 30D, Coateric, and Aquateric). The solubility of each of the above materials is either known or is readily determinable in vitro. The foregoing is a list of possible materials, but one of skill in the art with the benefit of the disclosure would recognize that it is not comprehensive and that there are other enteric materials that may be used.

Advantageously, kits are provided containing one or more compositions each including the same or different monomers. Such kits include a suitable dosage form such as those described above and instructions describing the method of using such dosage form to treat a disease or condition. The instructions would direct the consumer or medical personnel to administer the dosage form according to administration modes known to those skilled in the art. Such kits could advantageously be packaged and sold in single or multiple kit units. An example of such a kit is a so-called blister pack. Blister packs are well known in the packaging industry and are being widely used for the packaging of pharmaceutical unit dosage forms (tablets, capsules, and the like). Blister packs generally consist of a sheet of relatively stiff material covered with a foil of a preferably transparent plastic material. During the packaging process recesses are formed in the plastic foil. The recesses have the size and shape of the tablets or capsules to be packed. Next, the tablets or capsules are placed in the recesses and the sheet of relatively stiff material is sealed against the plastic foil at the face of the foil which is opposite from the direction in which the recesses were formed. As a result, the tablets or capsules are sealed in the recesses between the plastic foil and the sheet. Preferably the strength of the sheet is such that the tablets or capsules can be removed from the blister pack by manually applying pressure on the recesses whereby an opening is formed in the sheet at the place of the recess. The tablet or capsule can then be removed via said opening.

It may be desirable to provide a memory aid on the kit, e.g., in the form of numbers next to the tablets or capsules whereby the numbers correspond with the days of the regimen which the tablets or capsules so specified should be ingested. Another example of such a memory aid is a calendar printed on the card, e.g., as follows “First Week, Monday, Tuesday, . . . etc. . . . Second Week, Monday, Tuesday, . . . ” etc. Other variations of memory aids will be readily apparent. A “daily dose” can be a single tablet or capsule or several pills or capsules to be taken on a given day. Also, a daily dose of a first compound can consist of one tablet or capsule while a daily dose of the second compound can consist of several tablets or capsules and vice versa. The memory aid should reflect this.

Certain terms employed in the specification, examples, and appended claims are collected here. These definitions should be read in light of the entirety of the disclosure and understood as by a person of skill in the art. Unless defined otherwise, all technical and scientific terms used herein have the same meaning as commonly understood by a person of ordinary skill in the art.

### DEFINITIONS

In some embodiments, the compounds, as described herein, may be substituted with any number of substituents or functional moieties. In general, the term “substituted” whether preceded by the term “optionally” or not, and substituents contained in formulas, refer to the replacement of hydrogen radicals in a given structure with the radical of a specified substituent.

In some instances, when more than one position in any given structure may be substituted with more than one substituent selected from a specified group, the substituent may be either the same or different at every position.

As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. In some embodiments, heteroatoms such as nitrogen may have hydrogen substituents and/or any permissible substituents of organic compounds described herein which satisfy the valencies of the heteroatoms. Non-limiting examples of substituents include acyl; aliphatic; heteroaliphatic; aryl; heteroaryl; arylalkyl; heteroarylalkyl; alkoxy; cycloalkoxy; heterocyclylalkoxy; heterocyclyloxy; heterocyclyloxyalkyl; alkenyloxy; alkynyloxy; aryloxy; heteroalkoxy; heteroaryloxy; alkylthio; arylthio; heteroalkylthio; heteroarylthio; oxo; —F; —Cl; —Br; —I; —OH; —NO₂; —CN; —SCN; —SRₓ; —CF₃; —CH₂CF₃; —CHCl₂; —CH₂OH; —CH₂CH₂OH; —CH₂NH₂; —CH₂SO₂CH₃; —ORₓ, —C(O)Rₓ; —CO₂(Rₓ); —C(O)N(Rₓ)₂; —OC(O)Rₓ; —OCO₂Rₓ; —OC(O)N(Rₓ)₂; —N(Rₓ)₂; —SORₓ; —S(O)₂Rₓ; —NRₓC(O)Rₓ; or —C(Rₓ)₃; wherein each occurrence of Rₓ independently includes, but is not limited to, hydrogen, aliphatic, heteroaliphatic, aryl, heteroaryl, arylalkyl, or heteroarylalkyl, wherein any of the aliphatic, heteroaliphatic, arylalkyl, or heteroarylalkyl substituents described above and herein may be substituted or unsubstituted, branched or unbranched, cyclic or acyclic, and wherein any of the aryl or heteroaryl substituents described above and herein may be substituted or unsubstituted. Furthermore, the compounds described herein are not intended to be limited in any manner by the permissible substituents of organic compounds. In some embodiments, combinations of substituents and variables described herein may be preferably those that result in the formation of stable compounds. The term “stable,” as used herein, refers to compounds which possess stability sufficient to allow manufacture and which maintain the integrity of the compound for a sufficient period of time to be detected and preferably for a sufficient period of time to be useful for the purposes detailed herein.

The term “acyl,” as used herein, refers to a moiety that includes a carbonyl group. In some embodiments, an acyl group may have a general formula selected from —C(O)Rₓ; —CO₂(Rₓ); —C(O)N(Rₓ)₂; —OC(O)Rₓ; —OCO₂Rₓ; and —OC(O)N(Rₓ)₂; wherein each occurrence of Rₓ independently includes, but is not limited to, hydrogen, aliphatic, heteroaliphatic, aryl, heteroaryl, arylalkyl, or heteroarylalkyl, wherein any of the aliphatic, heteroaliphatic, arylalkyl, or heteroarylalkyl substituents described above and herein may be substituted or unsubstituted, branched or unbranched, cyclic or acyclic, and wherein any of the aryl or heteroaryl substituents described above and herein may be substituted or unsubstituted.

The term “aliphatic,” as used herein, includes both saturated and unsaturated, straight chain (i.e., unbranched), branched, acyclic, cyclic, or polycyclic aliphatic hydrocarbons, which are optionally substituted with one or more functional groups. As will be appreciated by one of ordinary skill in the art, “aliphatic” is intended herein to include, but is not limited to, alkyl, alkenyl, alkynyl, cycloalkyl, cycloalkenyl, and cycloalkynyl moieties. The term “heteroaliphatic,” as used herein, refers to aliphatic moieties that contain one or more oxygen, sulfur, nitrogen, phosphorus, or silicon atoms, e.g., in place of carbon atoms. Heteroaliphatic moieties may be branched, unbranched, cyclic or acyclic and include saturated and unsaturated heterocycles such as morpholino, pyrrolidinyl, etc. In certain embodiments, heteroaliphatic moieties are substituted by independent replacement of one or more of the hydrogen atoms thereon with one or more moieties including, but not limited to acyl; aliphatic; heteroaliphatic; aryl; heteroaryl; arylalkyl; heteroarylalkyl; alkoxy; cycloalkoxy; heterocyclylalkoxy; heterocyclyloxy; heterocyclyloxyalkyl; alkenyloxy; alkynyloxy; aryloxy; heteroalkoxy; heteroaryloxy; alkylthio; arylthio; heteroalkylthio; heteroarylthio; oxo; —F; —Cl; —Br; —I; —OH; —NO₂; —CN; —SCN; —SRₓ; —CF₃; —CH₂CF₃; —CHCl₂; —CH₂OH; —CH₂CH₂OH; —CH₂NH₂; —CH₂SO₂CH₃; —ORₓ, —C(O)Rₓ; —CO₂(Rₓ); —C(O)N(Rₓ)₂; —OC(O)Rₓ; —OCO₂Rₓ; —OC(O)N(Rₓ)₂; —N(Rₓ)₂; —SORₓ; —S(O)₂Rₓ; —NRₓC(O)Rₓ; or —C(Rₓ)₃; wherein each occurrence of Rₓ independently includes, but is not limited to, hydrogen, aliphatic, heteroaliphatic, aryl, heteroaryl, arylalkyl, or heteroarylalkyl, wherein any of the aliphatic, heteroaliphatic, arylalkyl, or heteroarylalkyl substituents described above and herein may be substituted or unsubstituted, branched or unbranched, cyclic or acyclic, and wherein any of the aryl or heteroaryl substituents described above and herein may be substituted or unsubstituted.

In general, the terms “aryl” and “heteroaryl,” as used herein, refer to stable mono- or polycyclic, heterocyclic, polycyclic, and polyheterocyclic unsaturated moieties having preferably 3-14 carbon atoms, each of which may be substituted or unsubstituted. Substituents include, but are not limited to, any of the previously mentioned substituents, i.e., the substituents recited for aliphatic moieties, or for other moieties as disclosed herein, resulting in the formation of a stable compound. In certain embodiments, aryl refers to a mono- or bicyclic carbocyclic ring system having one or two aromatic rings including, but not limited to, phenyl, naphthyl, tetrahydronaphthyl, indanyl, indenyl, and the like. In certain embodiments, the term heteroaryl, as used herein, refers to a cyclic aromatic radical having from five to ten ring atoms of which one ring atom is selected from the group consisting of S, O, and N; zero, one, or two ring atoms are additional heteroatoms independently selected from the group consisting of S, O, and N; and the remaining ring atoms are carbon, the radical being joined to the rest of the molecule via any of the ring atoms, such as, for example, pyridyl, pyrazinyl, pyrimidinyl, pyrrolyl, pyrazolyl, imidazolyl, thiazolyl, oxazolyl, isooxazolyl, thiadiazolyl, oxadiazolyl, thiophenyl, furanyl, quinolinyl, isoquinolinyl, and the like.

It will be appreciated that aryl and heteroaryl groups can be unsubstituted or substituted, wherein substitution includes replacement of one, two, three, or more of the hydrogen atoms thereon independently with any one or more of the following moieties including, but not limited to: aliphatic; heteroaliphatic; aryl; heteroaryl; arylalkyl; heteroarylalkyl; alkoxy; cycloalkoxy; heterocyclylalkoxy; heterocyclyloxy; heterocyclyloxyalkyl; alkenyloxy; alkynyloxy; aryloxy; heteroalkoxy; heteroaryloxy; alkylthio; arylthio; heteroalkylthio; heteroarylthio; oxo; —F; —Cl; —Br; —I; —OH; —NO₂; —CN; —CF₃; —CH₂CF₃; —CHCl₂; —CH₂OH; —CH₂CH₂OH; —CH₂NH₂; —CH₂SO₂CH₃; —C(O)Rₓ; —CO₂(Rₓ); —CON(Rₓ)₂; —OC(O)Rₓ; —OCO₂Rₓ; —OCON(Rₓ)₂; —N(Rₓ)₂; —S(O)₂Rₓ; —NRₓ(CO)Rₓ, wherein each occurrence of Rₓ independently includes, but is not limited to, hydrogen, aliphatic, heteroaliphatic, aryl, heteroaryl, arylalkyl, or heteroarylalkyl, wherein any of the aliphatic, heteroaliphatic, arylalkyl, or heteroarylalkyl substituents described above and herein may be substituted or unsubstituted, branched or unbranched, cyclic or acyclic, and wherein any of the aryl or heteroaryl substituents described above and herein may be substituted or unsubstituted. Additional examples of generally applicable substituents are illustrated by the specific embodiments shown in the Examples that are described herein.

The term “heterocyclic,” as used herein, refers to an aromatic or non-aromatic, partially unsaturated or fully saturated, 3- to 10-membered ring system, which includes single rings of 3 to 8 atoms in size and bi- and tri-cyclic ring systems which may include aromatic five- or six-membered aryl or aromatic heterocyclic groups fused to a non-aromatic ring. These heterocyclic rings include those having from one to three heteroatoms independently selected from the group consisting of oxygen, sulfur, and nitrogen, in which the nitrogen and sulfur heteroatoms may optionally be oxidized and the nitrogen heteroatom may optionally be quaternized. In certain embodiments, the term heterocyclic refers to a non-aromatic 5-, 6-, or 7-membered ring or a polycyclic group wherein at least one ring atom is a heteroatom selected from the group consisting of O, S, and N (wherein the nitrogen and sulfur heteroatoms may be optionally oxidized), including, but not limited to, a bi- or tri-cyclic group, comprising fused six-membered rings having between one and three heteroatoms independently selected from the group consisting of the oxygen, sulfur, and nitrogen, wherein (i) each 5-membered ring has 0 to 2 double bonds, each 6-membered ring has 0 to 2 double bonds, and each 7-membered ring has 0 to 3 double bonds, (ii) the nitrogen and sulfur heteroatoms may be optionally oxidized, (iii) the nitrogen heteroatom may optionally be quaternized, and (iv) any of the above heterocyclic rings may be fused to an aryl or heteroaryl ring.

The term “alkenyl” as used herein refers to an unsaturated straight or branched hydrocarbon having at least one carbon-carbon double bond, such as a straight or branched group of 2-6 or 3-4 carbon atoms, referred to herein for example as C₂₋₆alkenyl, and C₃₋₄alkenyl, respectively. Exemplary alkenyl groups include, but are not limited to, vinyl, allyl, butenyl, pentenyl, etc.

The term “alkenyloxy” used herein refers to a straight or branched alkenyl group attached to an oxygen (alkenyl-0). Exemplary alkenoxy groups include, but are not limited to, groups with an alkenyl group of 3-6 carbon atoms referred to herein as C₃₋₆alkenyloxy. Exemplary “alkenyloxy” groups include, but are not limited to allyloxy, butenyloxy, etc.

The term “alkoxy” as used herein refers to a straight or branched alkyl group attached to an oxygen (alkyl-O—). Exemplary alkoxy groups include, but are not limited to, groups with an alkyl group of 1-6 or 2-6 carbon atoms, referred to herein as C₁₋₆alkoxy, and C₂-C₆alkoxy, respectively. Exemplary alkoxy groups include, but are not limited to methoxy, ethoxy, isopropoxy, etc.

The term “alkoxycarbonyl” as used herein refers to a straight or branched alkyl group attached to oxygen, attached to a carbonyl group (alkyl-O—C(O)—). Exemplary alkoxycarbonyl groups include, but are not limited to, alkoxycarbonyl groups of 1-6 carbon atoms, referred to herein as C₁₋₆alkoxycarbonyl. Exemplary alkoxycarbonyl groups include, but are not limited to, methoxycarbonyl, ethoxycarbonyl, t-butoxycarbonyl, etc.

The term “alkynyloxy” used herein refers to a straight or branched alkynyl group attached to an oxygen (alkynyl-O)). Exemplary alkynyloxy groups include, but are not limited to, propynyloxy.

The term “alkyl” as used herein refers to a saturated straight or branched hydrocarbon, for example, such as a straight or branched group of 1-6, 1-4, or 1-3 carbon atoms, referred to herein as C₁₋₆alkyl, C₁₋₄alkyl, and C₁₋₃alkyl, respectively. Exemplary alkyl groups include, but are not limited to, methyl, ethyl, propyl, isopropyl, 2-methyl-1-propyl, 2-methyl-2-propyl, 2-methyl-1-butyl, 3-methyl-1-butyl, 3-methyl-2-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, butyl, isobutyl, t-butyl, pentyl, isopentyl, neopentyl, hexyl, etc.

The term “alkylcarbonyl” as used herein refers to a straight or branched alkyl group attached to a carbonyl group (alkyl-C(O)—). Exemplary alkylcarbonyl groups include, but are not limited to, alkylcarbonyl groups of 1-6 atoms, referred to herein as C₁₋₆alkylcarbonyl groups. Exemplary alkylcarbonyl groups include, but are not limited to, acetyl, propanoyl, isopropanoyl, butanoyl, etc.

The term “alkynyl” as used herein refers to an unsaturated straight or branched hydrocarbon having at least one carbon-carbon triple bond, such as a straight or branched group of 2-6, or 3-6 carbon atoms, referred to herein as C₂₋₆alkynyl, and C₃₋₆alkynyl, respectively. Exemplary alkynyl groups include, but are not limited to, ethynyl, propynyl, butynyl, pentynyl, hexynyl, methylpropynyl, etc.

The term “carbonyl” as used herein refers to the radical —C(O)—.

The term “carboxylic acid” as used herein refers to a group of formula —CO₂H.

The term “cyano” as used herein refers to the radical —CN.

The term “cycloalkoxy” as used herein refers to a cycloalkyl group attached to an oxygen (cycloalkyl-O—).

The term “cycloalkyl” as used herein refers to a monocyclic saturated or partially unsaturated hydrocarbon group of for example 3-6, or 4-6 carbons, referred to herein, e.g., as C₃₋₆cycloalkyl or C₄₋₆cycloalkyl and derived from a cycloalkane. Exemplary cycloalkyl groups include, but are not limited to, cyclohexyl, cyclohexenyl, cyclopentyl, cyclobutyl or, cyclopropyl.

The terms “halo” or “halogen” as used herein refer to F, Cl, Br, or I.

The term “heterocyclylalkoxy” as used herein refers to a heterocyclyl-alkyl-O— group.

The term “heterocyclyloxyalkyl” refers to a heterocyclyl-O-alkyl- group.

The term “heterocyclyloxy” refers to a heterocyclyl-O— group.

The term “heteroaryloxy” refers to a heteroaryl-O— group.

The terms “hydroxy” and “hydroxyl” as used herein refers to the radical —OH.

The term “oxo” as used herein refers to the radical ═O.

The term “connector” as used herein to refers to an atom or a collection of atoms optionally used to link interconnecting moieties, such as a disclosed linker and a pharmacophore. Contemplated connectors are generally hydrolytically stable.

“Treating” includes any effect, e.g., lessening, reducing, modulating, or eliminating, that results in the improvement of the condition, disease, disorder and the like.

“Pharmaceutically or pharmacologically acceptable” include molecular entities and compositions that do not produce an adverse, allergic, or other untoward reaction when administered to an animal, or a human, as appropriate. For human administration, preparations should meet sterility, pyrogenicity, general safety and purity standards as required by FDA Office of Biologics standards.

The term “pharmaceutically acceptable carrier” or “pharmaceutically acceptable excipient” as used herein refers to any and all solvents, dispersion media, coatings, isotonic and absorption delaying agents, and the like, that are compatible with pharmaceutical administration. The use of such media and agents for pharmaceutically active substances is well known in the art. The compositions may also contain other active compounds providing supplemental, additional, or enhanced therapeutic functions.

The term “pharmaceutical composition” as used herein refers to a composition comprising at least one compound as disclosed herein formulated together with one or more pharmaceutically acceptable carriers.

“Individual,” “patient,” or “subject” are used interchangeably and include any animal, including mammals, preferably mice, rats, other rodents, rabbits, dogs, cats, swine, cattle, sheep, horses, or primates, and most preferably humans. The compounds can be administered to a mammal, such as a human, but can also be administered to other mammals such as an animal in need of veterinary treatment, e.g., domestic animals (e.g., dogs, cats, and the like), farm animals (e.g., cows, sheep, pigs, horses, and the like) and laboratory animals (e.g., rats, mice, guinea pigs, and the like). The mammal treated is desirably a mammal in which treatment of obesity, or weight loss is desired. “Modulation” includes antagonism (e.g., inhibition), agonism, partial antagonism and/or partial agonism.

In the present specification, the term “therapeutically effective amount” means the amount of the subject compound that will elicit the biological or medical response of a tissue, system, animal, or human that is being sought by the researcher, veterinarian, medical doctor, or other clinician. The compounds are administered in therapeutically effective amounts to treat a disease. Alternatively, a therapeutically effective amount of a compound is the quantity required to achieve a desired therapeutic and/or prophylactic effect, such as an amount which results in weight loss.

The term “pharmaceutically acceptable salt(s)” as used herein refers to salts of acidic or basic groups that may be present in compounds used in the present compositions. Compounds included in the present compositions that are basic in nature are capable of forming a wide variety of salts with various inorganic and organic acids. The acids that may be used to prepare pharmaceutically acceptable acid addition salts of such basic compounds are those that form non-toxic acid addition salts, i.e., salts containing pharmacologically acceptable anions, including but not limited to malate, oxalate, chloride, bromide, iodide, nitrate, sulfate, bisulfate, phosphate, acid phosphate, isonicotinate, acetate, lactate, salicylate, citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate and pamoate (i.e., 1,1′-methylene-bis-(2-hydroxy-3-naphthoate)) salts. Compounds included in the present compositions that are acidic in nature are capable of forming base salts with various pharmacologically acceptable cations. Examples of such salts include alkali metal or alkaline earth metal salts and, particularly, calcium, magnesium, sodium, lithium, zinc, potassium, and iron salts. Compounds included in the present compositions that include a basic or acidic moiety may also form pharmaceutically acceptable salts with various amino acids. The compounds of the disclosure may contain both acidic and basic groups; for example, one amino and one carboxylic acid group. In such a case, the compound can exist as an acid addition salt, a zwitterion, or a base salt.

The compounds of the disclosure may contain one or more chiral centers and/or double bonds and, therefore, exist as stereoisomers, such as geometric isomers, enantiomers or diastereomers. The term “stereoisomers” when used herein consist of all geometric isomers, enantiomers or diastereomers. These compounds may be designated by the symbols “R” or “S,” depending on the configuration of substituents around the stereogenic carbon atom. Various stereoisomers of these compounds and mixtures thereof are encompassed by this disclosure. Stereoisomers include enantiomers and diastereomers. Mixtures of enantiomers or diastereomers may be designated “(±)” in nomenclature, but the skilled artisan will recognize that a structure may denote a chiral center implicitly.

The compounds of the disclosure may contain one or more chiral centers and/or double bonds and, therefore, exist as geometric isomers, enantiomers or diastereomers. The enantiomers and diastereomers may be designated by the symbols “(+),” “(−).” “R” or “S,” depending on the configuration of substituents around the stereogenic carbon atom, but the skilled artisan will recognize that a structure may denote a chiral center implicitly. Geometric isomers, resulting from the arrangement of substituents around a carbon-carbon double bond or arrangement of substituents around a cycloalkyl or heterocyclic ring, can also exist in the compounds. The symbol ═ denotes a bond that may be a single, double or triple bond as described herein. Substituents around a carbon-carbon double bond are designated as being in the “Z” or “E” configuration wherein the terms “Z” and “E” are used in accordance with IUPAC standards. Unless otherwise specified, structures depicting double bonds encompass both the “E” and “Z” isomers. Substituents around a carbon-carbon double bond alternatively can be referred to as “cis” or “trans,” where “cis” represents substituents on the same side of the double bond and “trans” represents substituents on opposite sides of the double bond. The arrangement of substituents around a carbocyclic ring can also be designated as “cis” or “trans.” The term “cis” represents substituents on the same side of the plane of the ring and the term “trans” represents substituents on opposite sides of the plane of the ring. Mixtures of compounds wherein the substituents are disposed on both the same and opposite sides of plane of the ring are designated “cis/trans.”

The term “stereoisomers” when used herein consist of all geometric isomers, enantiomers or diastereomers. Various stereoisomers of these compounds and mixtures thereof are encompassed by this disclosure.

Individual enantiomers and diasteriomers of the compounds can be prepared synthetically from commercially available starting materials that contain asymmetric or stereogenic centers, or by preparation of racemic mixtures followed by resolution methods well known to those of ordinary skill in the art. These methods of resolution are exemplified by (1) attachment of a mixture of enantiomers to a chiral auxiliary, separation of the resulting mixture of diastereomers by recrystallization or chromatography and liberation of the optically pure product from the auxiliary, (2) salt formation employing an optically active resolving agent, (3) direct separation of the mixture of optical enantiomers on chiral liquid chromatographic columns or (4) kinetic resolution using steroselective chemical or enzymatic reagents. Racemic mixtures can also be resolved into their component enantiomers by well known methods, such as chiral-phase gas chromatography or crystallizing the compound in a chiral solvent. Stereoselective syntheses, a chemical or enzymatic reaction in which a single reactant forms an unequal mixture of stereoisomers during the creation of a new stereocenter or during the transformation of a pre-existing one, are well known in the art. Stereoselective syntheses encompass both enantio- and diastereoselective transformations. For examples, see Carreira and Kvaerno, Classics in Stereoselective Synthesis, Wiley-VCH: Weinheim, 2009.

The compounds disclosed herein can exist in solvated as well as unsolvated forms with pharmaceutically acceptable solvents such as water, ethanol, and the like. In one embodiment, the compound is amorphous. In one embodiment, the compound is a polymorph. In another embodiment, the compound is in a crystalline form.

Also embraced are isotopically labeled compounds which are identical to those recited herein, except that one or more atoms are replaced by an atom having an atomic mass or mass number different from the atomic mass or mass number usually found in nature. Examples of isotopes that can be incorporated into the compounds include isotopes of hydrogen, carbon, nitrogen, oxygen, phosphorus, sulfur, fluorine and chlorine, such as ¹⁰B, ²H, ³H, ¹³C, ¹⁴C, ¹⁵N, ¹⁸O, ¹⁷O, ³¹P, ³²P, ³⁵S, ¹⁸F, and ³⁶Cl, respectively. For example, a compound may have one or more H atom replaced with deuterium.

Certain isotopically-labeled disclosed compounds (e.g., those labeled with ³H and ¹⁴C) are useful in compound and/or substrate tissue distribution assays. Tritiated (i.e., ³H) and carbon-14 (i.e., ¹⁴C) isotopes are particularly preferred for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. Isotopically labeled compounds can generally be prepared by following procedures analogous to those disclosed in the Examples herein by substituting an isotopically labeled reagent for a non-isotopically labeled reagent.

The term “prodrug” refers to compounds that are transformed in vivo to yield a disclosed compound or a pharmaceutically acceptable salt, hydrate or solvate of the compound. The transformation may occur by various mechanisms (such as by esterase, amidase, phosphatase, oxidative and or reductive metabolism) in various locations (such as in the intestinal lumen or upon transit of the intestine, blood, or liver). Prodrugs are well known in the art (for example, see Rautio, Kumpulainen, et al, Nature Reviews Drug Discovery 2008, 7, 255). For example, if a compound or a pharmaceutically acceptable salt, hydrate, or solvate of the compound contains a carboxylic acid functional group, a prodrug can comprise an ester formed by the replacement of the hydrogen atom of the acid group with a group such as (C₁₋₈)alkyl, (C₂₋₁₂)alkanoyloxymethyl, 1-(alkanoyloxy)ethyl having from 4 to 9 carbon atoms, 1-methyl-1-(alkanoyloxy)-ethyl having from 5 to 10 carbon atoms, alkoxycarbonyloxymethyl having from 3 to 6 carbon atoms, 1-(alkoxycarbonyloxy)ethyl having from 4 to 7 carbon atoms, 1-methyl-1-(alkoxycarbonyloxy)ethyl having from 5 to 8 carbon atoms, N-(alkoxycarbonyl)aminomethyl having from 3 to 9 carbon atoms, 1-(N-(alkoxycarbonyl)amino)ethyl having from 4 to 10 carbon atoms, 3-phthalidyl, 4-crotonolactonyl, gamma-butyrolacton-4-yl, di-N,N—(C₁-C₂)alkylamino(C₂-C₃)alkyl (such as β-dimethylaminoethyl), carbamoyl-(C₁-C₂)alkyl, N,N-di(C₁-C₂)alkylcarbamoyl-(C₁-C₂)alkyl and piperidino-, pyrrolidino- or morpholino(C₂-C₃)alkyl.

Similarly, if a compound contains an alcohol functional group, a prodrug can be formed by the replacement of the hydrogen atom of the alcohol group with a group such as (C₁₋₆)alkanoyloxymethyl, 1-((C₁₋₆)alkanoyloxy)ethyl, 1-methyl-1-((C₁₋₆)alkanoyloxy)ethyl (C₁₋₆)alkoxycarbonyloxymethyl, N—(C₁₋₆)alkoxycarbonylaminomethyl, succinoyl, (C₁₋₆)alkanoyl, α-amino(C₁₋₄alkanoyl, arylacyl and α-aminoacyl, or α-aminoacyl-α-aminoacyl, where each α-aminoacyl group is independently selected from the naturally occurring L-amino acids, P(O)(OH)₂, —P(O)(O(_{C1}-C₆)alkyl)₂ or glycosyl (the radical resulting from the removal of a hydroxyl group of the hemiacetal form of a carbohydrate).

If a compound incorporates an amine functional group, a prodrug can be formed, for example, by creation of an amide or carbamate, an N-acyloxyakyl derivative, an (oxodioxolenyl)methyl derivative, an N-Mannich base, imine, or enamine. In addition, a secondary amine can be metabolically cleaved to generate a bioactive primary amine, or a tertiary amine can be metabolically cleaved to generate a bioactive primary or secondary amine. For examples, see Simplicio, et al., Molecules 2008, 13, 519 and references therein.

### EXAMPLES

The invention is further illustrated by the following examples. The examples are provided for illustrative purposes only, and are not to be construed as limiting the scope or content of the invention in any way.

### Example 1

### Evaluation of a Rat Model of Heart Failure Treated with a Galectin-3 Inhibitor

In this study, N-acetyllactosamine (Gal3i) or an angiotensin-converting enzyme inhibitor (ACEi) was administered to TGR(mREN2)27 rats. Untreated TGR(mREN2)27 (REN2) and Sprague-Dawley (SD) rats were used as controls. TGR(mREN2)27 rats overexpress Ren-2, which leads to development of severe hypertension and ultimately heart failure. Fractional shortening, left ventricular end diastolic pressure (LVEDP), fibrosis, and survival were evaluated as a function of time.

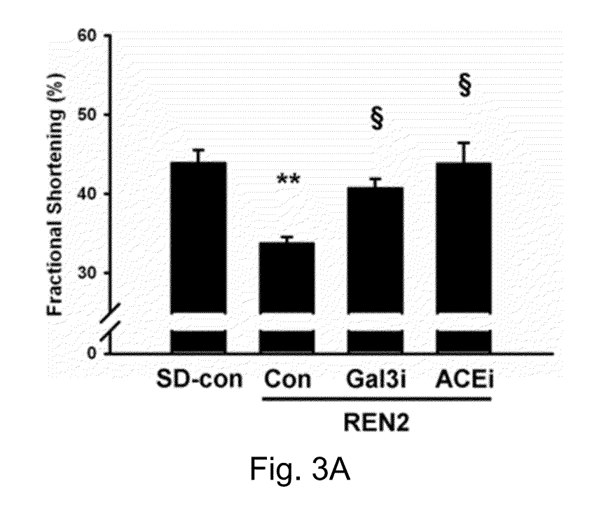

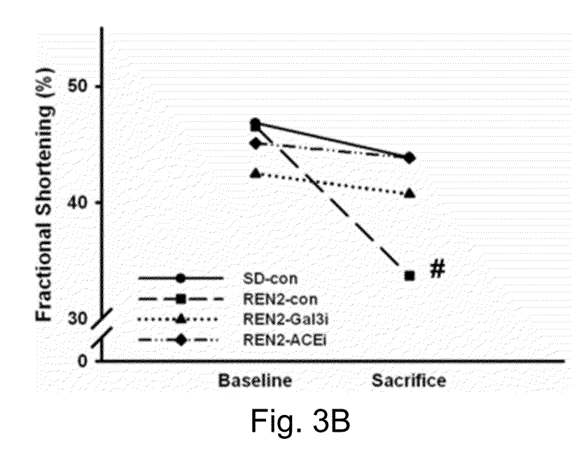

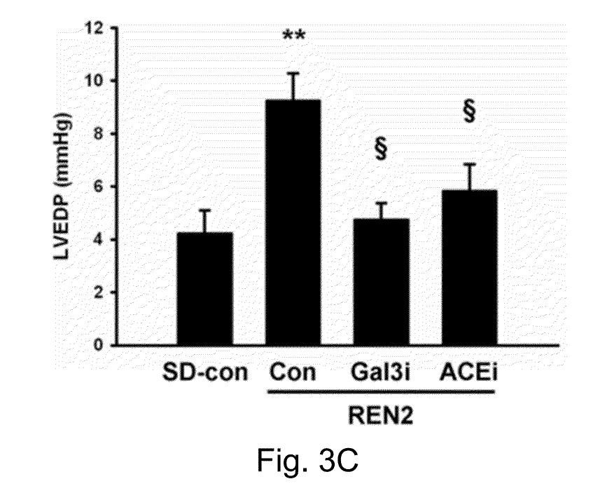

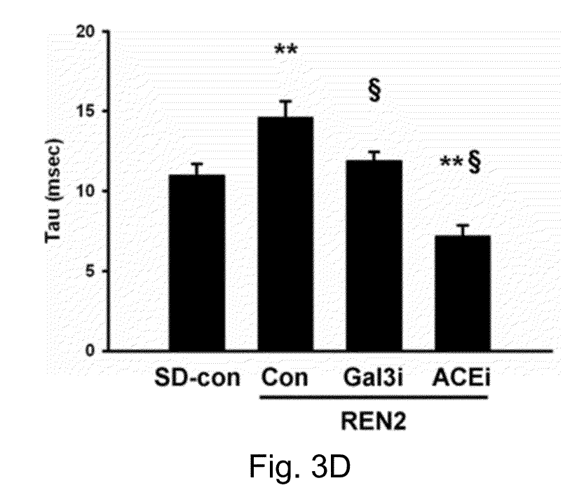

FIG. 3A shows a bar graph of fractional shortening for each study group of rats. Treatment with Gal3i or ACEi prevented a significant loss factional shortening relative to a placebo-treated REN2 control.

FIG. 3B shows a plot of fractional shortening at the beginning of the study and at sacrifice. Treatment with Gal3i or ACEi significantly slowed the reduction in fractional shortening relative to a placebo-treated REN2 control, and the rate of decrease in fractional shortening was comparable to that of the SD control group, which did not develop heart failure.

FIG. 3C shows a bar graph of LVEDP for each study group of rats. Treatment with Gal3i or ACEi significantly improved the LVEDP of the rats relative to a placebo-treated REN2 control.

FIG. 3D shows a bar graph of left ventricle pressure decay (Tau) for each study group of rats. Treatment with Gal3i or ACEi significantly improved the Tau of the rats relative to a placebo-treated REN2 control, and the Tau of the Gal3i-treated group was comparable to that of the SD control group.

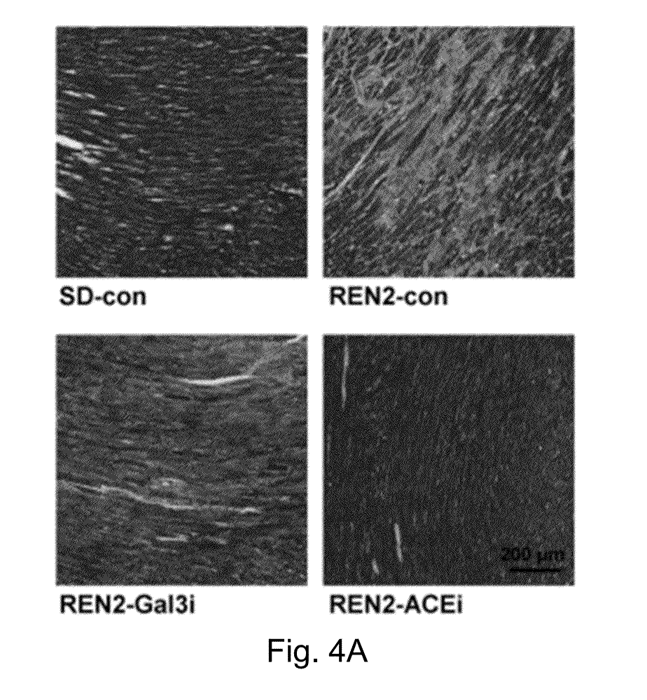

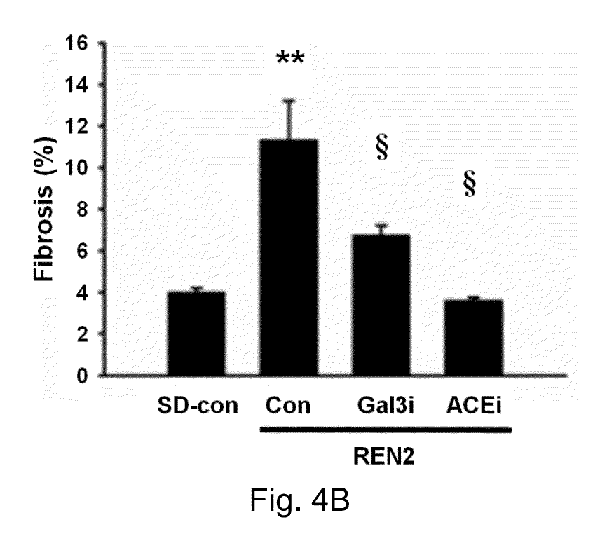

FIG. 4A shows cardiac tissue sections for each study group of rats. FIG. 4B shows a bar graph that quantifies the fibrosis for each study group of rats. As compared to a placebo-treated REN2 control group, administration of Gal3i or ACEi significantly inhibited fibrosis.

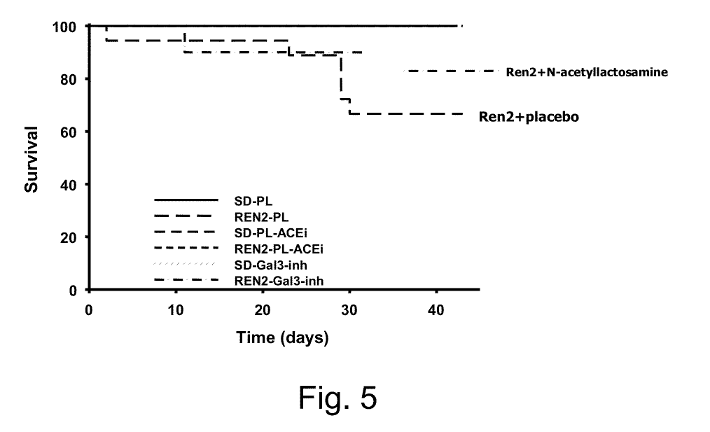

FIG. 5 shows a plot of study group survival as a function of time. The Kaplan-Meier curves in the plot indicate that approximately 80% of the Gal3i-treated rats and 90% of the ACEi-treated rats remained after 40 days, whereas only about 65% of the placebo-treated REN2 rats after the same time period.

### INCORPORATION BY REFERENCE

The entire disclosure of each of the patent documents and scientific articles referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes that come within the meaning and range of equivalency of the claims are intended to be embraced therein.

What is claimed is:

1. A method for treating heart failure in a patient, comprising:
administering to the patient a pharmaceutical composition comprising a carbohydrate in an amount sufficient to at least partially alleviate a symptom of heart failure, wherein the carbohydrate binds to galectin-3.

2. The method of claim 1, wherein the symptom of heart failure is selected from the group consisting of depressed fractional shortening, elevated left ventricular end diastolic pressure, elevated right ventricular end diastolic pressure, depressed left ventricular ejection fraction, depressed left ventricular end diastolic volume, depressed left ventricular right systolic volume, cardiac remodeling, and cardiac fibrosis.

3. A method for treating a patient at risk of developing heart failure, comprising:
administering to the patient a pharmaceutical composition comprising a carbohydrate in an amount sufficient to reduce the risk of developing heart failure, wherein the carbohydrate binds to galectin-3.

4. The method of claim 1, wherein the carbohydrate comprises a substituted lactosamine.

5. The method of claim 1, wherein the carbohydrate comprises N-acetyllactosamine.

6. The method of claim 1, wherein the carbohydrate comprises a purified pectin.

7. The method of claim 1, wherein the carbohydrate has a galectin-3 minimum inhibitory concentration less than about 200 μg/mL.

8. The method of claim 1, wherein the carbohydrate has a galectin-3 minimum inhibitory concentration less than about 50 μg/mL.

9.-10. (canceled)

11. A method for treating heart failure in a patient, comprising:
identifying the patient on the basis that the patient has elevated circulatory levels of galectin-3; and
administering to the patient a compound in an amount sufficient to at least partially inhibit decrease of, or to increase, left ventricular ejection fraction, wherein the compound binds galectin-3.

12.-13. (canceled)

14. A method for treating heart failure in a patient, comprising:
identifying the patient on the basis that the patient has elevated circulatory levels of galectin-3; and
administering to the patient a compound in an amount sufficient to at least partially inhibit increase of, or to decrease left ventricular end diastolic pressure, wherein the compound binds galectin-3.

15. A method for treating heart failure in a patient, comprising:
identifying the patient on the basis that the patient has elevated circulatory levels of galectin-3; and
administering to the patient a compound in an amount sufficient to at least partially inhibit cardiac remodeling, wherein the compound binds galectin-3.

16. A method for treating heart failure in a patient, comprising:
identifying the patient on the basis that the patient has elevated circulatory levels of galectin-3; and
administering to the patient a compound in an amount sufficient to at least partially inhibit cardiac fibrosis, wherein the compound binds galectin-3.

17.-26. (canceled)

27. A method of inhibiting galectin-3 in a patient in need thereof, comprising:
administering to the patient a composition comprising a carbohydrate and a pharmaceutically acceptable carrier, wherein the carbohydrate binds to galectin-3 and inhibits an activity of galectin-3; and
determining the activity of galectin-3 in the patient.

28. A method of inhibiting galectin-3 in a patient in need thereof, comprising:
administering to the patient an ingestible composition comprising a compound that binds galectin-3 and inhibits an activity of galectin-3; and
determining the activity of galectin-3 in the patient.

29.-38. (canceled)

39. A composition, comprising:
a purified pectin fragment and a pharmaceutically acceptable carrier, wherein the purified pectin fragment is capable of binding to galectin-3 and inhibiting an activity of galectin-3.

40. A method of inhibiting galectin-3 in a patient in need thereof, comprising:
administering to the patient a composition comprising a purified pectin fragment and a pharmaceutically acceptable carrier, wherein the purified pectin fragment binds to galectin-3 and inhibits an activity of galectin-3.

41.-59. (canceled)

60. A method of determining a galectin-3 inhibitory activity of a foodstuff, comprising:
administering the foodstuff to a subject; and
determining the activity of galectin-3 in the subject after administering the foodstuff to the subject.

61.-72. (canceled)

73. A method for providing information relating to galectin-3 inhibiting effects of a food product to a user, comprising:
providing a server having a network interface and hosting characteristics relating to a number of food products;
soliciting a user to input at least one food product characteristic to the server;
comparing the user input food product characteristic with the hosted food product characteristics;
selecting at least one hosted food product with characteristics comparable to the at least one user input characteristic; and
presenting to the user information relating to the galectin-3 inhibiting effects of the at least one selected food product.

74.-77. (canceled)

78. A method of selecting a therapy for a human, the method comprising measuring a galectin-3 blood concentration in a sample from the human, thereby to determine the presence or absence of a galectin-3 blood concentration indicative of responsiveness to an aldosterone antagonist.

79.-80. (canceled)

81. A method of treating a human comprising repeatedly administering an aldosterone antagonist to a patient having a determined galectin-3 blood concentration indicative of a survival-enhancing response to the aldosterone antagonist.

82.-103. (canceled)


SEQUENCE LISTING (10 sequences)

SEQ ID NO: 1 - protein, 12 aa, Homo sapiens
MADNFSLHDALS

SEQ ID NO: 2 - protein, 14 aa, Homo sapiens
MADNFSLHDALSGS

SEQ ID NO: 3 - protein, 11 aa, Homo sapiens
GNPNPQGWPGA

SEQ ID NO: 4 - protein, 10 aa, Homo sapiens
WGNQPAGAGG

SEQ ID NO: 5 - protein, 18 aa, Homo sapiens
YPGQAPPGAYPGQAPPGA

SEQ ID NO: 6 - protein, 16 aa, Homo sapiens
YPGAPGAYPGAPAPGV

SEQ ID NO: 7 - protein, 19 aa, Homo sapiens
YPGAPAPGVYPGPPSGPGA

SEQ ID NO: 8 - protein, 12 aa, Homo sapiens
YPSSGQPSATGA

SEQ ID NO: 9 - protein, 31 aa, Homo sapiens
YPGAPAPGVYPGPPSGPGAYPSSGQPSATGA

SEQ ID NO: 10 - protein, 4 aa, Unknown
  Bacterial species
CGPC